SEQUENCE LISTING (25 sequences)

SEQ ID NO: 1 - protein, 9 aa, Artificial Sequence
  CRE motif
LAGLIDADG

SEQ ID NO: 2 - DNA, 22 bp, Artificial Sequence
  CRE Recognition sequence
caaaacgtcgtgagacagtttg

SEQ ID NO: 3 - DNA, 978 bp, Homo sapiens
acctgaagagagaggaagctgtgggacaggtccagccagagcccaggccagaggggcagaagctggagtcctggagcacagacgcaggttgtcaggtgcacagagttggagggtcccctgctggtcatggttggccatgggggcacccaggagggatcagcgagctggctacacctaaaaatcagtccaaagtccgaggtccaaagagataaataaaaacattgacaaataagtcaaacggtccagcaacaaatggggacccaaggatcaggcacattgttttttttttttttggagtattagcatacactgtgacatgaaagagtgtgagctcaatcatgtggccttggactgggagagggcatttccttctcagaggcaactaagccctacctttgcctcagtctgggctctgtaatcctgctttgtgctcatctccccaagactgatcacctgggggaagctggggtggttcaaccctgcctgcccttccccaacatacagatggtggcaggtaacatggtgaaatcaaacagatctggtttttcatccgcctatccacccaggtgaccttggacaatttatttagcctctaggcctcagtagtcccagggaagtaataatcgctacttcactattttaaggattatataagatagggcatgtaaggtagctgacacacattcgcccatttcttcacgtgaattcagattctactgaattcagctctgcaccaagtcccatcctctgctgccctctgtttcagcaaacactgaggcctgttgtttctcctcctctaccggctctgatgcggtcaaggtccagagagctgagtagttggcaagcggaccaccagcaacagacaacatcttcattcggctctccctgaagctgtactgcctcgctgagaggatgaaggtctccgaggctgccctgtctctccttgtcctcatccttatcattacttcggcttctcgc

SEQ ID NO: 4 - DNA, 919 bp, Homo sapiens
tttgcctccaattcttccacagcacatagtgcatctcacataatatttataatgttggagtgcaattgtttatgtactctctcttacttactcaactataagatccttgaacttgtcacacacgttactttaggaactagcatggaaaaggtcttcacaaatactatctgaaaaatcaataagccagtaaaagtaaacctccatatgagcttactcctctgcttttcatcaccatctattctgtccacatttcaagcaatgtatttccagcatgaatttgtggtgtgatttgacatgaaggagaacatcagtcaccaaaatgaaaggattgtaccacagtacccttattttagtttaacttcgacttttttagattaactcttaatcaaaaattggtacttaacaaggaaaaacttgccttctgttgttttcagagaagtaaaaaaagcacctcacctttgagtcgccaaaattatgcttcctggaatgaccgcataccatgtagcaggtttattttagtttgtttacagaaaagtgtagaccaaagtccagtgctgactttgggagctactaaagacatgaaaaagtagaattttaaacacatttcattgtaaacgactgggagtatctgagcaaattatttcttacgtgactttagagaaaacggctacctatctgaccccaaaacgacttgaggaaactgtttccacggtcctgctgcagaggggaagcacagtcgtcaagaagagagtggggtcaggatcaaaacacatttagtgtgacttagggaaagaaaacattttccctctttgaacctctctggatacagtcattttgcctctacttgaggatcaactgttcaacctcaatggcctttcaggacctcctgggtcacgctggtgacctgtggagattccag

SEQ ID NO: 5 - DNA, 799 bp, Homo sapiens
ttggatctcagagacgggcaccgactgccgagccccagacgaagtgccccagggtcggggatcaggcttgtgcagggtagcacctggggacctgagggacctggaggccttcttgtatcctgaagtaggaccttctaagacttcatgagtcctggcccccttgcaggggttggggagctggggatcccagatctgcctattgcgcccgatgccccgaggctctctcttggactctggccctgagttcttctgcgcgatccttcggagacgtctggaggcctgctttatgcatctctcttggacctcagtttccccacacgtgggaggaggcagctggacgattcctgaaaggactttcccttgcttcctcatcacgtggaagagagcccacccggcacctggaaatggaaagccagtgaaggctgctttgggccggggcagcgggtgggaccgggcgggagggattccaaagagaccgccgggaaggctagagcttggaattccggctcctcggagtcctggccctcccccaccgccgcctcggagctcagcacaccttggatgggggaggcaggcagctcctagccccgcaccccaggaggcgcgctcggagggaagccgccaccgcgccgcctctgcctcggcgcggaacaaacggttaaagattttgggcagcgcctcgcggggggaggagccaggggcccaatccgcaattaaagatgaactttgggtgaactaattgtctgaccaaggtaacgtgggcagcaacctgggccgcctataaagcggcagcgccgtg

SEQ ID NO: 6 - DNA, 64 bp, Artificial Sequence
  constructed sequence
gagtgctctagttttgcaatacaggacatgctataaaaatggaaagatgttgctttctgagaga

SEQ ID NO: 7 - DNA, 113 bp, Artificial Sequence
  constructed sequence
ctttgaaaataccatcccagggttaatgctggggttaatttataactaagagtgctctagttttgcaatacaggacatgctataaaaatggaaagatgttgctttctgagaga

SEQ ID NO: 8 - DNA, 140 bp, Artificial Sequence
  constructed sequence
cctcttggccttggttttgtacatcagctttgaaaataccatcccagggttaatgctggggttaatttataactaagagtgctctagttttgcaatacaggacatgctataaaaatggaaagatgttgctttctgagaga

SEQ ID NO: 9 - DNA, 2729 bp, Artificial Sequence
  constructed sequence
  misc_feature (1)..(168): ITR
  misc_feature (206)..(1183): CCL16 promoter
  misc_feature (1193)..(2287): PCS7-8L.197 CDS
  misc_feature (2298)..(2512): PolyA
  misc_feature (2562)..(2729): ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagacctgaagagagaggaagctgtgggacaggtccagccagagcccaggccagaggggcagaagctggagtcctggagcacagacgcaggttgtcaggtgcacagagttggagggtcccctgctggtcatggttggccatgggggcacccaggagggatcagcgagctggctacacctaaaaatcagtccaaagtccgaggtccaaagagataaataaaaacattgacaaataagtcaaacggtccagcaacaaatggggacccaaggatcaggcacattgttttttttttttttggagtattagcatacactgtgacatgaaagagtgtgagctcaatcatgtggccttggactgggagagggcatttccttctcagaggcaactaagccctacctttgcctcagtctgggctctgtaatcctgctttgtgctcatctccccaagactgatcacctgggggaagctggggtggttcaaccctgcctgcccttccccaacatacagatggtggcaggtaacatggtgaaatcaaacagatctggtttttcatccgcctatccacccaggtgaccttggacaatttatttagcctctaggcctcagtagtcccagggaagtaataatcgctacttcactattttaaggattatataagatagggcatgtaaggtagctgacacacattcgcccatttcttcacgtgaattcagattctactgaattcagctctgcaccaagtcccatcctctgctgccctctgtttcagcaaacactgaggcctgttgtttctcctcctctaccggctctgatgcggtcaaggtccagagagctgagtagttggcaagcggaccaccagcaacagacaacatcttcattcggctctccctgaagctgtactgcctcgctgagaggatgaaggtctccgaggctgccctgtctctccttgtcctcatccttatcattacttcggcttctcgcgcggccgccatggcaccgaagaagaagcgcaaggtgcatatgaatacaaaatataataaagagttcttactctacttagcagggtttgtagacggtgacggttccatctttgccaggatcaagcctagtcaacgtagtaagttcaagcacaagctgcatctcgttttcgctgtctatcagaagacacagcgccgttggttcctcgacaagctggtggacgagatcggtgtgggttacgtgctggactctggcagcgtctccttttactcgctgtccgagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaagacgcgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatctatgcccgtatcaagccggttcagcgggctaagttcaagcacgagctggttctcgggttcgatgtcactcagaagacacagcgccgttggttcctcgacaagctggtggacgagatcggtgtgggttacgtgtatgacaagggcagcgtctccgcgtaccgtctgtcccagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtccccctaaggtacgatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 10 - DNA, 2669 bp, Artificial Sequence
  constructed sequence
  misc_feature (1)..(168): ITR
  misc_feature (206)..(1124): SLC22A9 promoter
  misc_feature (1133)..(2227): PCS7-8L CDS
  misc_feature (2238)..(2452): BGH Poly A
  misc_feature (2502)..(2669): ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagtttgcctccaattcttccacagcacatagtgcatctcacataatatttataatgttggagtgcaattgtttatgtactctctcttacttactcaactataagatccttgaacttgtcacacacgttactttaggaactagcatggaaaaggtcttcacaaatactatctgaaaaatcaataagccagtaaaagtaaacctccatatgagcttactcctctgcttttcatcaccatctattctgtccacatttcaagcaatgtatttccagcatgaatttgtggtgtgatttgacatgaaggagaacatcagtcaccaaaatgaaaggattgtaccacagtacccttattttagtttaacttcgacttttttagattaactcttaatcaaaaattggtacttaacaaggaaaaacttgccttctgttgttttcagagaagtaaaaaaagcacctcacctttgagtcgccaaaattatgcttcctggaatgaccgcataccatgtagcaggtttattttagtttgtttacagaaaagtgtagaccaaagtccagtgctgactttgggagctactaaagacatgaaaaagtagaattttaaacacatttcattgtaaacgactgggagtatctgagcaaattatttcttacgtgactttagagaaaacggctacctatctgaccccaaaacgacttgaggaaactgtttccacggtcctgctgcagaggggaagcacagtcgtcaagaagagagtggggtcaggatcaaaacacatttagtgtgacttagggaaagaaaacattttccctctttgaacctctctggatacagtcattttgcctctacttgaggatcaactgttcaacctcaatggcctttcaggacctcctgggtcacgctggtgacctgtggagattccagcggccgccatggcaccgaagaagaagcgcaaggtgcatatgaatacaaaatataataaagagttcttactctacttagcagggtttgtagacggtgacggttccatctttgccaggatcaagcctagtcaacgtagtaagttcaagcacaagctgcatctcgttttcgctgtctatcagaagacacagcgccgttggttcctcgacaagctggtggacgagatcggtgtgggttacgtgctggactctggcagcgtctccttttactcgctgtccgagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaagacgcgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatctatgcccgtatcaagccggttcagcgggctaagttcaagcacgagctggttctcgggttcgatgtcactcagaagacacagcgccgttggttcctcgacaagctggtggacgagatcggtgtgggttacgtgtatgacaagggcagcgtctccgcgtaccgtctgtcccagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtccccctaaggtacgatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 11 - DNA, 2549 bp, Artificial Sequence
  constructed sequence
  misc_feature (1)..(168): ITR
  misc_feature (206)..(1004): CYP26A1 - 1K promoter
  misc_feature (1013)..(2107): PCS7-8L CDS
  misc_feature (2118)..(2332): bGH Poly A
  misc_feature (2382)..(2549): ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagttggatctcagagacgggcaccgactgccgagccccagacgaagtgccccagggtcggggatcaggcttgtgcagggtagcacctggggacctgagggacctggaggccttcttgtatcctgaagtaggaccttctaagacttcatgagtcctggcccccttgcaggggttggggagctggggatcccagatctgcctattgcgcccgatgccccgaggctctctcttggactctggccctgagttcttctgcgcgatccttcggagacgtctggaggcctgctttatgcatctctcttggacctcagtttccccacacgtgggaggaggcagctggacgattcctgaaaggactttcccttgcttcctcatcacgtggaagagagcccacccggcacctggaaatggaaagccagtgaaggctgctttgggccggggcagcgggtgggaccgggcgggagggattccaaagagaccgccgggaaggctagagcttggaattccggctcctcggagtcctggccctcccccaccgccgcctcggagctcagcacaccttggatgggggaggcaggcagctcctagccccgcaccccaggaggcgcgctcggagggaagccgccaccgcgccgcctctgcctcggcgcggaacaaacggttaaagattttgggcagcgcctcgcggggggaggagccaggggcccaatccgcaattaaagatgaactttgggtgaactaattgtctgaccaaggtaacgtgggcagcaacctgggccgcctataaagcggcagcgccgtgcggccgccatggcaccgaagaagaagcgcaaggtgcatatgaatacaaaatataataaagagttcttactctacttagcagggtttgtagacggtgacggttccatctttgccaggatcaagcctagtcaacgtagtaagttcaagcacaagctgcatctcgttttcgctgtctatcagaagacacagcgccgttggttcctcgacaagctggtggacgagatcggtgtgggttacgtgctggactctggcagcgtctccttttactcgctgtccgagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaagacgcgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatctatgcccgtatcaagccggttcagcgggctaagttcaagcacgagctggttctcgggttcgatgtcactcagaagacacagcgccgttggttcctcgacaagctggtggacgagatcggtgtgggttacgtgtatgacaagggcagcgtctccgcgtaccgtctgtcccagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtccccctaaggtacgatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 12 - DNA, 1817 bp, Artificial Sequence
  constructed sequence
  misc_feature (1)..(168): ITR
  misc_feature (206)..(269): TBG-S1-F64 promoter
  misc_feature (281)..(1375): PCS7-8 CDS
  misc_feature (1386)..(1600): bGH Poly A
  misc_feature (1650)..(1817): ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaaggagtgctctagttttgcaatacaggacatgctataaaaatggaaagatgttgctttctgagagacagcggccgccatggcaccgaagaagaagcgcaaggtgcatatgaatacaaaatataataaagagttcttactctacttagcagggtttgtagacggtgacggttccatctttgccaggatcaagcctagtcaacgtagtaagttcaagcacaagctgcatctcgttttcgctgtctatcagaagacacagcgccgttggttcctcgacaagctggtggacgagatcggtgtgggttacgtgctggactctggcagcgtctccttttactcgctgtccgagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaagacgcgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatctatgcccgtatcaagccggttcagcgggctaagttcaagcacgagctggttctcgggttcgatgtcactcagaagacacagcgccgttggttcctcgacaagctggtggacgagatcggtgtgggttacgtgtatgacaagggcagcgtctccgcgtaccgtctgtcccagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtccccctaaggtacgatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 13 - DNA, 1866 bp, Artificial Sequence
  constructed sequence
  misc_feature (1)..(168): ITR
  misc_feature (206)..(318): TBG-S1-F113 promoter
  misc_feature (330)..(1424): PCS7-8 CDS
  misc_feature (1435)..(1649): bGH Poly A
  misc_feature (1699)..(1866): ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctttgaaaataccatcccagggttaatgctggggttaatttataactaagagtgctctagttttgcaatacaggacatgctataaaaatggaaagatgttgctttctgagagacagcggccgccatggcaccgaagaagaagcgcaaggtgcatatgaatacaaaatataataaagagttcttactctacttagcagggtttgtagacggtgacggttccatctttgccaggatcaagcctagtcaacgtagtaagttcaagcacaagctgcatctcgttttcgctgtctatcagaagacacagcgccgttggttcctcgacaagctggtggacgagatcggtgtgggttacgtgctggactctggcagcgtctccttttactcgctgtccgagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaagacgcgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatctatgcccgtatcaagccggttcagcgggctaagttcaagcacgagctggttctcgggttcgatgtcactcagaagacacagcgccgttggttcctcgacaagctggtggacgagatcggtgtgggttacgtgtatgacaagggcagcgtctccgcgtaccgtctgtcccagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtccccctaaggtacgatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 14 - DNA, 1893 bp, Artificial Sequence
  constructed sequence
  misc_feature (1)..(168): ITR
  misc_feature (206)..(345): TBG-S1-F140
  misc_feature (357)..(1451): PCS7-8 CDS
  misc_feature (1462)..(1676): bGH PolyA
  misc_feature (1726)..(1893): ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagcctcttggccttggttttgtacatcagctttgaaaataccatcccagggttaatgctggggttaatttataactaagagtgctctagttttgcaatacaggacatgctataaaaatggaaagatgttgctttctgagagacagcggccgccatggcaccgaagaagaagcgcaaggtgcatatgaatacaaaatataataaagagttcttactctacttagcagggtttgtagacggtgacggttccatctttgccaggatcaagcctagtcaacgtagtaagttcaagcacaagctgcatctcgttttcgctgtctatcagaagacacagcgccgttggttcctcgacaagctggtggacgagatcggtgtgggttacgtgctggactctggcagcgtctccttttactcgctgtccgagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaagacgcgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatctatgcccgtatcaagccggttcagcgggctaagttcaagcacgagctggttctcgggttcgatgtcactcagaagacacagcgccgttggttcctcgacaagctggtggacgagatcggtgtgggttacgtgtatgacaagggcagcgtctccgcgtaccgtctgtcccagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtccccctaaggtacgatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 15 - DNA, 2625 bp, Artificial sequence
  PCSK7-8L.197
  CDS (1089)..(2183): M2PCSK9 coding sequence
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagactgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggcaccgaagaagaagcgcaaggtgcatatgaatacaaaatataataaagagttcttactctacttagcagggtttgtagacggtgacggttccatctttgccaggatcaagcctagtcaacgtagtaagttcaagcacaagctgcatctcgttttcgctgtctatcagaagacacagcgccgttggttcctcgacaagctggtggacgagatcggtgtgggttacgtgctggactctggcagcgtctccttttactcgctgtccgagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaagacgcgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatctatgcccgtatcaagccggttcagcgggctaagttcaagcacgagctggttctcgggttcgatgtcactcagaagacacagcgccgttggttcctcgacaagctggtggacgagatcggtgtgggttacgtgtatgacaagggcagcgtctccgcgtaccgtctgtcccagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtccccctaaggtacgatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 16 - protein, 364 aa, Artificial sequence
  Synthetic Construct
MAPKKKRKVHMNTKYNKEFLLYLAGFVDGDGSIFARIKPSQRSKFKHKLHLVFAVYQKTQRRWFLDKLVDEIGVGYVLDSGSVSFYSLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYARIKPVQRAKFKHELVLGFDVTQKTQRRWFLDKLVDEIGVGYVYDKGSVSAYRLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 17 - DNA, 2214 bp, adeno-associated virus rh79
  CDS (1)..(2214)
atggctgctgacggttatcttccagattggctcgaggacaacctctctgagggcattcgcgagtggtgggacctgaaacctggagcccccaagcccaaggccaaccagcagaagcaggacgacggccggggtctggtgcttcctggctacaagtacctcggacccttcaacggactcgacaagggggagcccgtcaacgaggcggacgccgcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaagctgctaagacggctcctggaaagaagagaccggtagaaccgtcacctcagcgatcccccgactcctccacgggcatcggcaaaaaaggccagcagcccgcgagaaagagactgaactttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctgcccacctacaacaaccacctctacaagcaaatctccaatgggacatcgggaggaagcaccaacgacaacacctacttcggctacagcaccccctgggggtattttgacttcaacagattccactgtcacttctcaccacgtgactggcagagactcatcaacaacaactggggattccggcccaagagactcagcttcaagctcttcaacatccaggttaaggaggtcacgcagaatgaaggcaccaagaccatcgccaataaccttaccagcacgattcaggtatttacggactcggaataccagctgccgtacgtcctcggctccgcgcaccagggctgcctgcctccgttcccggcggatgtcttcatgattccccagtacggctacctgacactgaacaacggaagtcaagccgtaggccgttcctcattctactgcctggaatattttccatctcaaatgctgcggactggaaacaactttgaatttagctacacctttgaggacgtgcccttccacagcagctacgcacacagccagagcctggaccggctgatgaaccctctcatcgaccagtacctgtattacctatccagaactcagtccacaggaggaactcaaggtacacagcaattgttattttctcaagccgggcctgcaaatatgtcggctcaggccaagaactggctacctggaccttgctaccggcagcagcgagtctccacgacactgtcgcaaaacaacaacagcaactttgcttggactggtgccacgaaatatcatctgaacggaagagactctttggtgaatcccggtgttgctatggcaacgcataaggacgacgaggaacgtttctttccatcgagcggagtcctgatgtttggaaaacagggtgctggaagagacaatgtggactatagcagcgttatgctaaccagcgaggaagaaatcaagaccactaaccctgtagccactgaacaatacggcgtggtggctgataacttgcagcaaaccaatacaggacctatcgtgggaaatgtcaacagccaaggagccttacctggcatggtctggcagaaccgagacgtgtacctgcagggtcccatttgggccaagattcctcacacggacggcaactttcacccgtctcctctgatgggcggctttggactgaaacacccgcctcctcaaatcctgatcaagaacactcccgttcctgcggatcctccaacgacgttcagccaggcgaaattggcttccttcatcacgcagtatagtaccggccaggtcagcgtggagatcgagtgggagctgcagaaggagaacagcaagcgctggaacccagaaattcagtatacttccaactactacaaatctacaaatgtggactttgctgtcaataccgagggtacatattcagagcctcgccccattggaactcgttacctcacccgtaatctg

SEQ ID NO: 18 - protein, 738 aa, adeno-associated virus rh79
MAADGYLPDWLEDNLSEGIREWWDLKPGAPKPKANQQKQDDGRGLVLPGYKYLGPFNGLDKGEPVNEADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPARKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 19 - DNA, 2214 bp, adeno-associated virus 8
  CDS (1)..(2214)
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctg

SEQ ID NO: 20 - protein, 738 aa, adeno-associated virus 8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 21 - DNA, 2214 bp, adeno-associated virus hu37
  CDS (1)..(2214)
atggctgctgacggttatcttccagattggctcgaggacaacctctctgagggcattcgcgagtggtgggacctgaaacctggagcccccaagcccaaggccaaccagcagaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaagctgctaagacggctcctggaaagaagagaccggtagaaccgtcacctcagcgttcccccgactcctccacgggcatcggcaagaaaggccagcagcccgctaaaaagagactgaactttggtcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctgcccacctacaacaaccacctctacaagcaaatatccaatgggacatcgggaggaagcaccaacgacaacacctacttcggctacagcaccccctgggggtattttgacttcaacagattccactgccacttctcaccacgtgactggcagcgactcatcaacaacaactggggattccggccaaaaagactcagcttcaagctcttcaacatccaggtcaaggaggtcacgcagaatgaaggcaccaagaccatcgccaataaccttaccagcacgattcaggtatttacggactcggaataccagctgccgtacgtcctcggctccgcgcaccagggctgcctgcctccgttcccggcggacgtcttcatgattccccagtacggctaccttacactgaacaatggaagtcaagccgtaggccgttcctccttctactgcctggaatattttccatctcaaatgctgcgaactggaaacaattttgaattcagctacaccttcgaggacgtgcctttccacagcagctacgcacacagccagagcttggaccgactgatgaatcctctcatcgaccagtacctgtactacttatccagaactcagtccacaggaggaactcaaggtacccagcaattgttattttctcaagctgggcctgcaaacatgtcggctcaggctaagaactggctacctggaccttgctaccggcagcagcgagtctctacgacactgtcgcaaaacaacaacagcaactttgcttggactggtgccaccaaatatcacctgaacggaagagactctttggtaaatcccggtgtcgccatggcaacccacaaggacgacgaggaacgcttcttcccgtcgagtggagtcctgatgttcggaaaacagggtgctggaagagacaatgtggactacagcagcgttatgctaaccagcgaagaagaaattaaaaccactaaccccgtagccacagaacaatacggtgtggtggctgacaacttgcagcaaaccaatacagggcctattgtgggaaatgtcaacagccaaggagccttacctggcatggtctggcagaaccgagacgtgtacctgcagggtcccatctgggccaagattcctcacacggacggcaacttccacccttcaccgctaatgggaggatttggactgaagcacccacctcctcagatcctgatcaagaacacgccggtacctgcggatcctccaacaacgttcagccaggcgaaattggcttccttcattacgcagtacagcaccggacaggtcagcgtggaaatcgagtgggagctgcagaaggagaacagcaaacgctggaacccagagattcagtacacttcaaactactacaaatctacaaatgtggactttgctgtcaatacagagggaacttattctgagcctcgccccattggtactcgttacctcacccgtaatctg

SEQ ID NO: 22 - protein, 738 aa, adeno-associated virus hu37
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 23 - DNA, 2217 bp, adeno-associated virus rh.90
  CDS (1)..(2217)
atggctgccgatggttatcttccagattggctcgaggacaacctctctgagggcattcgcgagtggtgggacctgaaacctggagccccaaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgataatccgtacctgcggtataaccacgccgacgccgagtttcaggagcgtctgcaagaagatacgtcatttgggggcaacctcgggcgagcagtcttccaggccaagaagcgggttctcgaacctctcggtctggttgaggaaggcgctaagacggctcctggaaagaagagaccggtagagccatcaccacagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacaatggctgcaggcggtggcgcaccaatggcagacaataacgaaggtgccgacggagtgggtagttcctcgggaaattggcattgcgattccacatggctgggcgacagagtcatcaccaccagcacccgaacctgggccctgcccacctacaacaaccacctctacaagcaaatctccaacgggacctcgggaggcagcaccaacgacaacacctacttcggctacagcaccccctgggggtattttgactttaacagattccactgccacttctcaccacgtgactggcagcgacttatcaacaacaactggggattccggcccaagagactcagcttcaagctcttcaacatccaggtcaaggaggtcacgcaaaatgaaggcaccaagaccatcgccaataacctcaccagcaccatccaggtgtttacggactcggaataccagctgccatacgtcctcggctctgcccaccagggctgcctgcctccgttcccggcggacgtcttcatgattcctcagtatggctacctgacgctgaacaacggaagtcaggccgtgggccgttcctccttctactgcctggagtactttccctctcagatgctaagaacgggcaacaacttctccttcagctataccttcgaggacgtgcctttccacagcagctacgcgcacagccagagcctggaccggctgatgaatcccctcattgaccagtacctgtactacctgtcgcggacacaatccacaggaggcacagcgggaactcagcagttgctgttttctcaggccgggcctaacaacatgtctgctcaggccaaaaactggctacccggaccttgttatcggcagcaacgtgtttccacgacactgtcgcaaaacaacaacagcaactttgcctggaccggtgccaccaaataccacctgaacggaagagactctctggtaaatccgggtgtcgccatggcaaccaacaaggacgacgaggaccgcttcttcccatccagcggcatcctcatgtttggcaagcagggggctggaaaagacaacgtggactatagcaacgtgatgctaaccagcgaggaagaaatcaagaccactaaccctgtggccacagaacagtatggtgtggtggcggataacctgcagcagcaaaacacagctcctattgtgggggccgtcaacagccaaggagccttacctggcatggtttggcagaaccgggacgtgtacctgcagggtcccatctgggccaagattcctcacacggatggtaactttcacccgtctcctctcatgggcggctttggacttaaacatccgcctcctcagatcctgatcaagaacactcccgttcctgcggatcctccaacggcgttcaaccaggccaagctgaactctttcatcacgcagtacagcaccggacaagtcagcgtggagatcgagtgggagctgcagaaggagaacagcaagcgctggaacccagagattcagtatacctccaactactacaaatctacaaatgtggactttgctgttaatactgagggtgtttactctgagcctcgccccattggcacccgttacctcacccgtaatctgtaa

SEQ ID NO: 24 - protein, 738 aa, adeno-associated virus rh.90
MAADGYLPDWLEDNLSEGIREWWDLKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTEGVYSEPRPIGTRYLTRNL

SEQ ID NO: 25 - DNA, 176 bp, Artificial Sequence
  constructed sequence
actcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagaga

(19) United States
(12) Patent Application Publication
(10) Pub. No.: US 2023/0167464 A1
(43) Pub. Date: Jun. 1, 2023

(54) COMPOSITIONS AND METHODS FOR REDUCING NUCLEASE EXPRESSION AND OFF-TARGET ACTIVITY USING A PROMOTER WITH LOW TRANSCRIPTIONAL ACTIVITY

(71) Applicant: The Trustees of the University of Pennsylvania, Philadelphia, PA (US)

(72) Inventors: Camilo Breton, St. Davids, PA (US); James M. Wilson, Philadelphia, PA (US)

(21) Appl. No.: 17/997,108

(22) PCT Filed: Apr. 27, 2021
(86) PCT No.: PCT/US21/29403

§ 371 Date: Oct. 25, 2022

Related U.S. Application Data
(60) Provisional application No. 63/016,139, filed on Apr. 27, 2020.

(51) Int. Cl. C12N 15/90; C12N 15/861; C12N 9/22

(52) U.S. Cl. CPC C12N 15/907; C12N 15/861; C12N 9/22; C12N 2750/14143; C12N 2830/00

(57) ABSTRACT

A gene editing nuclease expression cassette is provided which comprises a nucleic acid sequence comprising a nuclease coding sequence which is operably linked to regulatory sequences which direct expression of the nuclease following delivery to a host cell, wherein the regulatory sequences comprise a weak promoter. A vector is provided comprising the gene editing nuclease expression cassette. Also provided are compositions containing same and methods of use.

### BACKGROUND OF THE INVENTION

The use of engineered nucleases has been described for editing dysfunctional genes. AAV-mediated delivery of such nucleases has also been described. However, while AAV-mediated delivery of nucleases avoids the need for repeated readministration, the resulting nuclease it is continuously expressed in the target tissue following vector transduction, which may induce immune responses and cellular toxicity.

Further, both in vitro and in vivo studies have shown that nucleases, regardless of delivery vehicle, generate indels (insertions and deletions) in other regions of the genome, suggesting off-target activity. This off-target activity is undesirable and, especially for clinical studies, it is imperative to reduce or eliminate this off-target activity, while retaining the high on-target efficacy.

What are needed are improved compositions and methods for gene editing.

### SUMMARY OF THE INVENTION

In one aspect, a gene targeting nuclease expression cassette is provided. In one embodiment, the expression cassette includes a nucleic acid comprising a nuclease coding sequence which is operably linked to regulatory sequences which direct expression of the nuclease following delivery to a host cell having a sequence to which the nuclease is targeted, wherein the regulatory sequences comprise a promoter which has low transcriptional activity. In one embodiment, the promoter is a liver-specific promoter. In another embodiment, the promoter is a TBG-S1 promoter variant. In yet another embodiment, the promoter is TBG-S1-F64. In another embodiment, the promoter is TBG-S1-F113. In another embodiment, the promoter is TBG-S1-F140. In another embodiment, the promoter is a CCL16 promoter. In another embodiment, the promoter is a SCLC22A9 promoter. In another embodiment, the promoter is a CYP26A1 promoter. In yet another embodiment, the nuclease is a meganuclease, a CRISPR/Cas nuclease, zinc finger nuclease, or TALEN.

In another aspect, a recombinant AAV useful for gene editing is provided. The rAAV includes an AAV capsid and a vector genome packaged in the AAV capsid, wherein the vector genome includes an expression cassette as described herein, and AAV inverted terminal repeats required for packaging the expression cassette into the capsid.

In another aspect, a method for editing a targeted gene is provided. The method includes delivering a nuclease expression cassette, a composition, or a viral vector according as described herein, to a subject.

In another aspect, a method for reducing off-target activity of a gene targeting nuclease is provided. The method includes delivering a nuclease expression cassette, a composition, or a viral vector according as described herein, to a subject.

In another aspect, a novel “weak promoter” is provided. In another embodiment, the promoter is TBG-S1-F64. In another embodiment, the promoter is TBG-S1-F113. In another embodiment, the promoter is TBG-S1-F140. In another embodiment, the promoter comprises the sequence of SEQ ID NO: 6. In another embodiment, the promoter comprises the sequence of SEQ ID NO: 7. In another embodiment, the promoter comprises the sequence of SEQ ID NO: 8.

In another aspect, a pharmaceutical composition comprising a nuclease expression cassette, a composition, or a viral vector according as described herein is provided. The composition includes one or more of a carrier, suspending agent, and/or excipient.

Other aspects and advantages of the invention will be apparent from the following detailed description of the invention.

### BRIEF DESCRIPTION OF THE DRAWINGS

FIG. 1 is a schematic representation of AAV constructs containing “weak” promoters for vectors used in Example 1 (data shown in FIGS. 2-5). Promoter: Shortened versions of human Thyroxine-binding Globulin (TBG) gene or derived from the promoter sequence of liver-enriched genes: CCL16, CYP26A1, or SLC22A9 (identified using Human Protein Atlas database). M2PCSK9: Engineered I-CreI meganuclease targeting a 22 bp sequence in the human PCSK9 gene (sometimes referred to as the ARCUS meganuclease). PolyA: Bovine growth hormone polyadenylation signal.

FIG. 2A shows the levels at 7 weeks post-AAV of indels in the region corresponding to the target sequence of the ARCUS nuclease, quantified by a next-generation sequencing assay. Linear scale.

FIG. 2B shows the same levels as FIG. 2A, logarithmic scale.

FIG. 2C shows average levels at week 9 of recombinant PCSK9 in serum, determined by an ELISA assay, per treated group.

FIG. 3 shows the number of off-target loci in the genomic DNA as a result of the nuclease activity as determined using an NGS-based method called ITR-Seq.

FIG. 4 shows the indels in a set of genomic locations corresponding to the identified off-targets. Indels levels for each off-target are shown relative to the indels levels in TBG control group (arbitrary value of 1).

FIG. 5 shows the hPCSK9 levels at 7 weeks after treatment (as a percentage of baseline) for vectors tested in Example 1.

FIGS. 6-10 show data for the NHP pilot study described in Example 2. NHPs were injected with 6×10¹² GS/kg of the indicated vectors. Liver biopsies were performed at day 18 and 128, and DNA/RNA analysis done to detect on-target and off-target genome editing by next generation sequencing. A summary of some of the data presented in FIGS. 7-10 is shown in FIG. 6. Indel % (FIG. 7) and number of off-targets (FIG. 8) were determined in DNA from liver biopsies at day 18 post-AAV. PCSK9 levels (as a percentage of baseline) are shown in FIG. 9 (for 7 weeks post-AAV). LDL levels (as a percentage of baseline) are shown in FIG. 10.

FIG. 11 is a schematic of the NHP Pharmaceutical/Toxicity Study design described in Example 3.

FIG. 12 is an alignment of the sequences of TBG-S1 promoter and F64, F113, and F140 promoters described herein.

### DETAILED DESCRIPTION OF THE INVENTION

The compositions and methods provided herein are designed to produce lower expression of, or minimize off-target activity of a persistently expressed enzyme (e.g., following delivery of an expression cassette) and/or modulating the activity of the expressed enzyme. Use of these compositions and methods with non-secreting enzymes which may accumulate in a cell and/or enzymes which accumulate at higher than desired levels prior to secretion is particularly desirable. The compositions and methods of the invention are well suited for use with gene editing enzymes, particularly meganucleases. However, other applications will be apparent to one of skill in the art.

### Low-Transcription Promoters (“Weak” Promoters)

In one aspect, a novel promoter having low-transcriptional activity, or weak promoter, is provided. As used herein, the term “promoter having low-transcriptional activity” or “weak promoter” refers to an expression control sequence which produces a low level of expression of the coding sequence. In one embodiment, the term “low-transcriptional activity” refers to a level of transcription less than the level induced by a reference “strong promoter”. In one embodiment, the reference strong promoter is the thyroxin binding globulin (TBG) promoter or TBG-S1 promoter. Other reference “strong” promoters are known in the art.

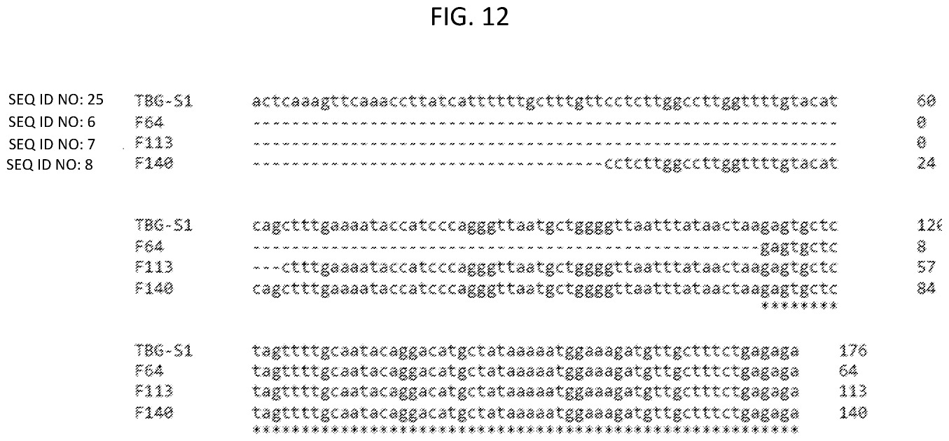

In one embodiment, the promoter is a weakened version of the liver-specific thyroxin binding globulin (TBG) promoter. In one embodiment, the weak promoter is truncated at the 5′ or 3′ end of the native promoter, or TBG-S1 sequence. In one embodiment, the promoter retains only the 3′ terminal 64 nt from the TBG-S1 promoter, and is termed F64 (also called TBG-S1-F64) (SEQ ID NO: 6). In another embodiment, the promoter retains only the 3′ terminal 113 nt from the TBG-S1 promoter and is termed F113 (also called TBG-S1-F113) (SEQ ID NO: 7). In one embodiment, the promoter retains only the 3′ terminal 140 nt from the TBG-S1 promoter and is termed F140 (also called TBG-S1-F140) (SEQ ID NO: 8). An alignment of the TBG-S1, F64, F113 and F140 sequences is shown in FIG. 12. In one embodiment, the promoter shares at least 90%, 95%, 96%, 97%, 98%, 99% or 99.9% identity with SEQ ID NO: 6. In one embodiment, the promoter shares at least 90%, 95%, 96%, 97%, 98%, 99% or 99.9% identity with SEQ ID NO: 7. In one embodiment, the promoter shares at least 90%, 95%, 96%, 97%, 98%, 99% or 99.9% identity with SEQ ID NO: 8.

In other embodiments, weak promoters useful herein include known promoters. In one embodiment, the weak promoter is the CCL16 promoter (SEQ ID NO: 3). In another embodiment, the weak promoter is the SLC22A9 promoter (SEQ ID NO: 4). In yet another embodiment, the weak promoter is the CYP26A1 promoter (SEQ ID NO: 5).

### Expression Cassettes and Vectors

In another aspect, an expression cassette is provided. In one embodiment, the expression cassette includes a weak promoter, as described herein, operably linked to a coding sequence. In one embodiment, the expression cassette includes the coding sequence for a nuclease under the control of regulatory sequences which comprise a promoter having low-transcriptional activity, as described herein. In another aspect, vectors comprising the expression cassette (and promoter) are provided.

The examples herein illustrate use of AAV vectors containing the promoter having low-transcriptional activity (weak promoter) in the vector genome. However, the use of weak promoters is not limited to AAV constructs and can be used for other vectors. In certain embodiments, the vector genome may be packaged into a different vector (e.g., a recombinant bocavirus). In certain embodiments, the expression cassette may be packaged into a different viral vector, into a non-viral vector, and/or into a different delivery system. Suitably, the coding sequence for a transgene is engineered into an expression cassette, operably linked to regulatory elements which include the weak promoter in the cell containing the target site for the enzyme.

As used herein, an “expression cassette” refers to a nucleic acid molecule which comprises a coding sequence (or transgene), promoter, and may include other regulatory sequences therefor, which cassette may be engineered into a genetic element and/or packaged into the capsid of a viral vector (e.g., a viral particle). Typically, such an expression cassette for generating a viral vector contains the sequences described herein flanked by packaging signals of the viral genome and other expression control sequences such as those described herein. The transgene is a nucleic acid sequence, heterologous to the vector sequences flanking the transgene, which encodes a polypeptide, protein, or other product, of interest. The nucleic acid coding sequence is operatively linked to regulatory components in a manner which permits transgene transcription, translation, and/or expression in a target cell. The heterologous nucleic acid sequence (transgene) can be derived from any organism. The AAV may comprise one or more transgenes. Exemplified herein is the use of the weak promoters described herein in conjunction with a gene editing nuclease (specifically, a meganuclease). However, the weak promoters may be incorporated into any expression cassette where lower expression and/or a short promoter sequence is desired.

In one embodiment, the coding sequence encodes a nuclease selected from a meganuclease, a zinc finger nuclease, a transcription activator-like (TAL) effector nuclease (TALEN), and a clustered, regularly interspaced short palindromic repeat (CRISPR)/endonuclease (Cas9, Cpf1, etc). Examples of suitable meganucleases are described, e.g., in U.S. Pat. Nos. 8,445,251; 9,340,777; 9,434,931; 9,683,257, and WO 2018/195449. Other suitable enzymes include nuclease-inactive S. pyogenes CRISPR/Cas9 that can bind RNA in a nucleic-acid-programmed manner (Nelles et al, Programmable RNA Tracking in Live Cells with CRISPR/Cas9, Cell, 165(2):P488-96 (April 2016)), and base editors (e.g., Levy et al. Cytosine and adenine base editing of the brain, liver, retina, heart and skeletal muscle of mice via adeno-associated viruses, Nature Biomedical Engineering, 4, 97-110 (January 2020)). In certain embodiments, the nuclease is not a zinc finger nuclease. In certain embodiments, the nuclease is not a CRISPR-associated nuclease. In certain embodiments, the nuclease is not a TALEN. In one embodiment, the nuclease is not a meganuclease.

In certain embodiments, the nuclease is a member of the LAGLIDADG (SEQ ID NO: 1) family of homing endonucleases. In certain embodiments, the nuclease is a member of the I-CreI family of homing endonucleases which recognizes and cuts a 22 base pair recognition sequence SEQ ID NO: 2—CAAAACGTCGTGAGACAGTTTG. See, e.g., WO 2009/059195. Methods for rationally-designing mono-LAGLIDADG homing endonucleases were described which are capable of comprehensively redesigning ICreI and other homing endonucleases to target widely-divergent DNA sites, including sites in mammalian, yeast, plant, bacterial, and viral genomes (WO 2007/047859). In one embodiment, the nuclease is not the nuclease encoded by the sequence shown in nt 1089 to 2183 of SEQ ID NO: 15. In one embodiment, the nuclease is not the protein sequence shown in SEQ ID NO: 16.

One of the aims of the invention is to reduce the off-target activity of a nuclease without compromising its strong on-target activity. It was hypothesized that high expression of the nuclease in transduced cells is not needed to achieve editing of the target DNA sequence, and that the off-target results from an elevated accumulation of the nuclease in the cell. To reduce nuclease expression, high-expressing promoters were replaced by promoters with lower transcriptional activity. Thus, the expression cassette contains a promoter sequence as part of the expression control sequences or the regulatory sequences. As described herein, the promoter is a promoter having lower transcriptional activity, or “weak promoter”.

In one embodiment, the weak promoter is the CCL16 promoter (SEQ ID NO: 3). In another embodiment, the weak promoter is the SLC22A9 promoter (SEQ ID NO: 4). In yet another embodiment, the weak promoter is the CYP26A1 promoter (SEQ ID NO: 5).

In addition, in another embodiment, the promoter is a weakened version of a tissue-specific promoter. In one example, the tissue-specific promoter is the liver-specific thyroxin binding globulin (TBG) promoter. In one embodiment, the weak promoter is truncated at the 5′ or 3′ end of the native promoter, or TBG-S1 sequence. In one embodiment, the promoter retains only the 3′ terminal 64 nt from the TBG-S1 promoter, and is termed F64 (SEQ ID NO: 6). In one embodiment, the promoter retains only the 3′ terminal 113 nt from the TBG-S1 promoter and is termed F113 (SEQ ID NO: 7). In one embodiment, the promoter retains only the 3′ terminal 140 nt from the TBG-S1 promoter and is termed F140 (SEQ ID NO: 8).

In addition to a promoter, the expression cassette and/or a vector may contain one or more appropriate “regulatory elements” or “regulatory sequences”, which comprise but are not limited to an enhancer; transcription factor; transcription terminator; efficient RNA processing signals such as splicing and polyadenylation signals (polyA); sequences that stabilize cytoplasmic mRNA, for example Woodchuck Hepatitis Virus (WHP) Posttranscriptional Regulatory Element (WPRE); sequences that enhance translation efficiency (i.e., Kozak consensus sequence); sequences that enhance protein stability; and when desired, sequences that enhance secretion of the encoded product. Examples of suitable polyA sequences include, e.g., SV40, bovine growth hormone (bGH), and TK polyA. Examples of suitable enhancers include, e.g., the alpha fetoprotein enhancer, the TTR minimal promoter/enhancer, LSP (TH-binding globulin promoter/alpha1-microglobulin/bikunin enhancer), amongst others. These control sequences or the regulatory sequences are operably linked to the nuclease coding sequences.

In certain embodiments, the weak promoters, constructs containing same and methods described herein, are useful in targeting liver-directed therapies. Thus, liver-expressed genes are useful herein as well as nucleases targeted to these genes, or sequences flanking the same. Liver-expressed genes include, without limitation, proprotein convertase subtilisin/kexin type 9 (PCSK9) (cholesterol related disorders), transthyretin (TTR) (transthyretin amyloidosis), HAO, apolipoprotein C-III (APOC3), Factor VIII, Factor IX, low density lipoprotein receptor (LDLr), lipoprotein lipase (LPL) (Lipoprotein Lipase Deficiency), lecithin-cholesterol acyltransferase (LCAT), ornithine transcarbamylase (OTC), carnosinase (CN1), sphingomyelin phosphodiesterase (SMPD1) (Niemann-Pick disease), hypoxanthine-guanine phosphoribosyltransferase (HGPRT), branched-chain alpha-keto acid dehydrogenase complex (BCKDC) (maple syrup urine disease), erythropoietin (EPO), Carbamyl Phosphate Synthetase (CPS1), N-Acetylglutamate Synthetase (NAGS), Argininosuccinic Acid Synthetase (Citrullinemia), Argininosuccinate Lyase (ASL) (Argininosuccinic Aciduria), and Arginase (AG).

In certain embodiments, the rAAV may be used in gene editing systems, which system may involve one rAAV or co-administration of multiple rAAV stocks. For example, the rAAV may be engineered to deliver SpCas9, SaCas9, ARCUS, Cpf1, and other suitable gene editing constructs.

In one embodiment, a nucleic acid molecule is provided which encodes a PCSK9 meganuclease operably linked to a weak promoter. In one embodiment, the weak promoter is F64. In another embodiment, the weak promoter is F113. In yet another embodiment, the weak promoter is F140. In another embodiment, the weak promoter is the CCL16 promoter. In another embodiment, the weak promoter is the SLC22A9 promoter. In yet another embodiment, the weak promoter is the CYP26A1 promoter. In certain embodiments, a meganuclease may be selected from those described in WO 2018/195449A1. In one embodiment, the nucleic acid molecule comprises the F113 promoter operably linked to the PCSK9 meganuclease coding sequence of nt 1089 to 2183 of SEQ ID NO: 15, or a sequence sharing at least 95%, 96%, 97%, 98%, 99%, or 99.9% identity thereto. In one embodiment, the nucleic acid molecule comprises the F113 promoter operably linked to the sequence encoding the PCSK9 meganuclease of SEQ ID NO: 16, or a sequence sharing at least 95%, 96%, 97%, 98%, 99%, or 99.9% identity thereto.

In another embodiment, the nucleic acid molecule comprises the F64 promoter operably linked to the PCSK9 meganuclease coding sequence of nt 1089 to 2183 of SEQ ID NO: 15, or a sequence sharing at least 95%, 96%, 97%, 98%, 99%, or 99.9% identity thereto. In one embodiment, the nucleic acid molecule comprises the F64 promoter operably linked to the sequence encoding the PCSK9 meganuclease of SEQ ID NO: 16, or a sequence sharing at least 95%, 96%, 97%, 98%, 99%, or 99.9% identity thereto.

In another embodiment, the nucleic acid molecule comprises the F140 promoter operably linked to the PCSK9 meganuclease coding sequence of nt 1089 to 2183 of SEQ ID NO: 15, or a sequence sharing at least 95%, 96%, 97%, 98%, 99%, or 99.9% identity thereto. In one embodiment, the nucleic acid molecule comprises the F140 promoter operably linked to the sequence encoding the PCSK9 meganuclease of SEQ ID NO: 16, or a sequence sharing at least 95%, 96%, 97%, 98%, 99%, or 99.9% identity thereto.

In another embodiment, the nucleic acid molecule comprises the SLC22A9 promoter operably linked to the PCSK9 meganuclease coding sequence of nt 1089 to 2183 of SEQ ID NO: 15, or a sequence sharing at least 95%, 96%, 97%, 98%, 99%, or 99.9% identity thereto. In one embodiment, the nucleic acid molecule comprises the SLC22A9 promoter operably linked to the sequence encoding the PCSK9 meganuclease of SEQ ID NO: 16, or a sequence sharing at least 95%, 96%, 97%, 98%, 99%, or 99.9% identity thereto.

In another embodiment, the nucleic acid molecule comprises the CCL16 promoter operably linked to the PCSK9 meganuclease coding sequence of nt 1089 to 2183 of SEQ ID NO: 15, or a sequence sharing at least 95%, 96%, 97%, 98%, 99%, or 99.9% identity thereto. In one embodiment, the nucleic acid molecule comprises the CCL16 promoter operably linked to the sequence encoding the PCSK9 meganuclease of SEQ ID NO: 16, or a sequence sharing at least 95%, 96%, 97%, 98%, 99%, or 99.9% identity thereto.

In another embodiment, the nucleic acid molecule comprises the CYP26A1 promoter operably linked to the PCSK9 meganuclease coding sequence of nt 1089 to 2183 of SEQ ID NO: 15, or a sequence sharing at least 95% to 99.9% identity thereto. In one embodiment, the nucleic acid molecule comprises the CYP26A1 promoter operably linked to the sequence encoding the PCSK9 meganuclease of SEQ ID NO: 16, or a sequence sharing at least 95%, 96%, 97%, 98%, 99%, or 99.9% identity thereto.

In one embodiment, a nucleic acid molecule is provided which encodes a TTR meganuclease operably linked to a weak promoter. In one embodiment, the weak promoter is F64. In another embodiment, the weak promoter is F113. In yet another embodiment, the weak promoter is F140. In another embodiment, the weak promoter is the CCL16 promoter. In another embodiment, the weak promoter is the SLC22A9 promoter. In yet another embodiment, the weak promoter is the CYP26A1 promoter.

In one embodiment, a nucleic acid molecule is provided which encodes a HAO meganuclease operably linked to a weak promoter. In one embodiment, the weak promoter is F64. In another embodiment, the weak promoter is F113. In yet another embodiment, the weak promoter is F140. In another embodiment, the weak promoter is the CCL16 promoter. In another embodiment, the weak promoter is the SLC22A9 promoter. In yet another embodiment, the weak promoter is the CYP26A1 promoter.

In one embodiment, a nucleic acid molecule is provided which encodes a BCKDC meganuclease operably linked to a weak promoter. In one embodiment, the weak promoter is F64. In another embodiment, the weak promoter is F113. In yet another embodiment, the weak promoter is F140. In another embodiment, the weak promoter is the CCL16 promoter. In another embodiment, the weak promoter is the SLC22A9 promoter. In yet another embodiment, the weak promoter is the CYP26A1 promoter.

In one embodiment, a nucleic acid molecule is provided which encodes an APOC3 meganuclease operably linked to a weak promoter. In one embodiment, the weak promoter is F64. In another embodiment, the weak promoter is F113. In yet another embodiment, the weak promoter is F140. In another embodiment, the weak promoter is the CCL16 promoter. In another embodiment, the weak promoter is the SLC22A9 promoter. In yet another embodiment, the weak promoter is the CYP26A1 promoter.

In one embodiment, a nucleic acid molecule is provided which encodes a CRISPR/Cas9 nuclease operably linked to a weak promoter. In one embodiment, the weak promoter is F64. In another embodiment, the weak promoter is F113. In yet another embodiment, the weak promoter is F140. In another embodiment, the weak promoter is the CCL16 promoter. In another embodiment, the weak promoter is the SLC22A9 promoter. In yet another embodiment, the weak promoter is the CYP26A1 promoter. In one embodiment, the promoters, cassettes and rAAV described herein are useful in the CRISPR-Cas dual vector system described in WO 2016/176191 which is incorporated herein by reference.

In another embodiment, the transgene is selected for use in gene correction therapy. This may be accomplished using, e.g., a zinc-finger nuclease (ZFN)-induced DNA double-strand break in conjunction with an exogenous DNA donor substrate. See, e.g., Ellis et al, Gene Therapy (epub January 2012) 20:35-42 which is incorporated herein by reference. The transgenes may be readily selected by one of skill in the art based on the desired result.

In one embodiment, a nucleic acid molecule is provided which encodes a zinc finger nuclease operably linked to a weak promoter. In one embodiment, the weak promoter is F64. In another embodiment, the weak promoter is F113. In yet another embodiment, the weak promoter is F140. In another embodiment, the weak promoter is the CCL16 promoter. In another embodiment, the weak promoter is the SLC22A9 promoter. In yet another embodiment, the weak promoter is the CYP26A1 promoter.

In one embodiment, a nucleic acid molecule is provided which encodes a transcription activator-like effector nuclease (TALEN) operably linked to a weak promoter. In one embodiment, the weak promoter is F64. In another embodiment, the weak promoter is F113. In yet another embodiment, the weak promoter is F140. In another embodiment, the weak promoter is the CCL16 promoter. In another embodiment, the weak promoter is the SLC22A9 promoter. In yet another embodiment, the weak promoter is the CYP26A1 promoter.

Other useful products encoded by the transgene include a variety of gene products which replace a defective or deficient gene, inactivate or “knock-out”, or “knock-down” or reduce the expression of a gene which is expressing at an undesirably high level, or delivering a gene product which has a desired therapeutic effect. In some embodiments, the therapy will be “somatic gene therapy”, i.e., transfer of genes to a cell of the body which does not produce sperm or eggs. In certain embodiments, the transgenes express proteins have the sequence of native human sequences. However, in other embodiments, synthetic proteins are expressed. Such proteins may be intended for treatment of humans, or in other embodiments, designed for treatment of animals, including companion animals such as canine or feline populations, or for treatment of livestock or other animals which come into contact with human populations.

Examples of suitable gene products may include those associated with familial hypercholesterolemia, muscular dystrophy, cystic fibrosis, and rare or orphan diseases. Examples of such rare disease may include spinal muscular atrophy (SMA), Huntingdon's Disease, Rett Syndrome (e.g., methyl-CpG-binding protein 2 (MeCP2); UniProtKB—P51608), Amyotrophic Lateral Sclerosis (ALS), Duchenne Type Muscular dystrophy, Friedrichs Ataxia (e.g., frataxin), ATXN2 associated with spinocerebellar ataxia type 2 (SCA2)/ALS; TDP-43 associated with ALS, progranulin (PRGN) (associated with non-Alzheimer's cerebral degenerations, including, frontotemporal dementia (FTD), progressive non-fluent aphasia (PNFA) and semantic dementia), among others. See, e.g., www.orpha.net/consor/cgi-bin/Disease_Search_List.php; rarediseases.info.nih.gov/diseases.

Examples of suitable genes may include, e.g., hormones and growth and differentiation factors including, without limitation, insulin, glucagon, glucagon-like peptide-1 (GLP1), growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO) (including, e.g., human, canine or feline epo), connective tissue growth factor (CTGF), neutrophic factors including, e.g., basic fibroblast growth factor (bFGF), acidic fibroblast growth factor (aFGF), epidermal growth factor (EGF), platelet-derived growth factor (PDGF), insulin growth factors I and II (IGF-I and IGF-II), any one of the transforming growth factor α superfamily, including TGFα, activins, inhibins, or any of the bone morphogenic proteins (BMP) BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

Other useful transgene products include proteins that regulate the immune system including, without limitation, cytokines and lymphokines such as thrombopoietin (TPO), interleukins (IL) IL-1 through IL-36 (including, e.g., human interleukins IL-1, IL-1α, IL-1β, IL-2, IL-3, IL-4, IL-6, IL-8, IL-12, IL-11, IL-12, IL-13, IL-18, IL-31, IL-35), monocyte chemoattractant protein, leukemia inhibitory factor, granulocyte-macrophage colony stimulating factor, Fas ligand, tumor necrosis factors α and β, interferons α, β, and γ, stem cell factor, flk-2/flt3 ligand. Gene products produced by the immune system are also useful in the invention. These include, without limitations, immunoglobulins IgG, IgM, IgA, IgD and IgE, chimeric immunoglobulins, humanized antibodies, single chain antibodies, T cell receptors, chimeric T cell receptors, single chain T cell receptors, class I and class II MHC molecules, as well as engineered immunoglobulins and MHC molecules. For example, in certain embodiments, the rAAV antibodies may be designed to delivery canine or feline antibodies, e.g., such as anti-IgE, anti-IL31, anti-IL33, anti-CD20, anti-NGF, anti-GnRH. Useful gene products also include complement regulatory proteins such as complement regulatory proteins, membrane cofactor protein (MCP), decay accelerating factor (DAF), CR1, CF2, CD59, and C1 esterase inhibitor (C1-INH).

Still other useful gene products include any one of the receptors for the hormones, growth factors, cytokines, lymphokines, regulatory proteins and immune system proteins. The invention encompasses receptors for cholesterol regulation and/or lipid modulation, including the low density lipoprotein (LDL) receptor, high density lipoprotein (HDL) receptor, the very low density lipoprotein (VLDL) receptor, and scavenger receptors. The invention also encompasses gene products such as members of the steroid hormone receptor superfamily including glucocorticoid receptors and estrogen receptors, Vitamin D receptors and other nuclear receptors. In addition, useful gene products include transcription factors such as jun, fos, max, mad, serum response factor (SRF), AP-1, AP2, myb, MyoD and myogenin, ETS-box containing proteins, TFE3, E2F, ATF1, ATF2, ATF3, ATF4, ZFS, NFAT, CREB, HNF-4, C/EBP, SP1, CCAAT-box binding proteins, interferon regulation factor (IRF-1), Wilms tumor protein, ETS-binding protein, STAT, GATA-box binding proteins, e.g., GATA-3, and the forkhead family of winged helix proteins.

Other useful gene products include hydroxymethylbilane synthase (HMBS), carbamoyl synthetase I, ornithine transcarbamylase (OTC), arginosuccinate synthetase, arginosuccinate lyase (ASL) for treatment of argunosuccinate lyase deficiency, arginase, fumarylacetate hydrolase, phenylalanine hydroxylase, alpha-1 antitrypsin, rhesus alpha-fetoprotein (AFP), rhesus chorionic gonadotrophin (CG), glucose-6-phosphatase, porphobilinogen deaminase, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, and a dystrophin gene product [e.g., a mini- or micro-dystrophin]. Still other useful gene products include enzymes such as may be useful in enzyme replacement therapy, which is useful in a variety of conditions resulting from deficient activity of enzyme. For example, enzymes that contain mannose-6-phosphate may be utilized in therapies for lysosomal storage diseases (e.g., a suitable gene includes that encoding β-glucuronidase (GUSB)). In another example, the gene product is ubiquitin protein ligase E3A (UBE3A). Still useful gene products include UDP Glucuronosyltransferase Family 1 Member A1 (UGT1A1).

Still other useful gene products include those used for treatment of hemophilia, including hemophilia B (including Factor IX) and hemophilia A (including Factor VIII and its variants, such as the light chain and heavy chain of the heterodimer and the B-deleted domain; U.S. Pat. Nos. 6,200,560 and 6,221,349). In some embodiments, the minigene comprises first 57 base pairs of the Factor VIII heavy chain which encodes the 10 amino acid signal sequence, as well as the human growth hormone (hGH) polyadenylation sequence. In alternative embodiments, the minigene further comprises the A1 and A2 domains, as well as 5 amino acids from the N-terminus of the B domain, and/or 85 amino acids of the C-terminus of the B domain, as well as the A3, C1 and C2 domains. In yet other embodiments, the nucleic acids encoding Factor VIII heavy chain and light chain are provided in a single minigene separated by 42 nucleic acids coding for 14 amino acids of the B domain [U.S. Pat. No. 6,200,560].

Further illustrative genes which may be delivered via the rAAV include, without limitation, glucose-6-phosphatase, associated with glycogen storage disease or deficiency type 1A (GSD1), phosphoenolpyruvate-carboxykinase (PEPCK), associated with PEPCK deficiency; cyclin-dependent kinase-like 5 (CDKL5), also known as serine/threonine kinase 9 (STK9) associated with seizures and severe neurodevelopmental impairment; galactose-1 phosphate uridyl transferase, associated with galactosemia; phenylalanine hydroxylase (PAH), associated with phenylketonuria (PKU); gene products associated with Primary Hyperoxaluria Type 1 including Hydroxyacid Oxidase 1 (GO/HAO1) and AGXT, branched chain alpha-ketoacid dehydrogenase, including BCKDH, BCKDH-E2, BAKDH-E1a, and BAKDH-E1b, associated with Maple syrup urine disease; fumarylacetoacetate hydrolase, associated with tyrosinemia type 1; methylmalonyl-CoA mutase, associated with methylmalonic acidemia; medium chain acyl CoA dehydrogenase, associated with medium chain acetyl CoA deficiency; ornithine transcarbamylase (OTC), associated with ornithine transcarbamylase deficiency; argininosuccinic acid synthetase (ASS1), associated with citrullinemia, lecithin-cholesterol acyltransferase (LCAT) deficiency; amethylmalonic acidemia (MMA); NPC1 associated with Niemann-Pick disease, type C1); propionic academia (PA); TTR associated with Transthyretin (TTR)-related Hereditary Amyloidosis; low density lipoprotein receptor (LDLR) protein, associated with familial hypercholesterolemia (FH), LDLR variant, such as those described in WO 2015/164778; PCSK9; ApoE and ApoC proteins, associated with dementia; UDP-glucouronosyltransferase, associated with Crigler-Najjar disease; adenosine deaminase, associated with severe combined immunodeficiency disease; hypoxanthine guanine phosphoribosyl transferase, associated with Gout and Lesch-Nyan syndrome; biotimidase, associated with biotimidase deficiency; alpha-galactosidase A (a-Gal A) associated with Fabry disease); beta-galactosidase (GLB1) associated with GM1 gangliosidosis; ATP7B associated with Wilson's Disease; beta-glucocerebrosidase, associated with Gaucher disease type 2 and 3; peroxisome membrane protein 70 kDa, associated with Zellweger syndrome; arylsulfatase A (ARSA) associated with metachromatic leukodystrophy, galactocerebrosidase (GALC) enzyme associated with Krabbe disease, alpha-glucosidase (GAA) associated with Pompe disease; sphingomyelinase (SMPD1) gene associated with Nieman Pick disease type A; argininosuccsinate synthase associated with adult onset type II citrullinemia (CTLN2); carbamoyl-phosphate synthase 1 (CPS1) associated with urea cycle disorders; survival motor neuron (SMN) protein, associated with spinal muscular atrophy; ceramidase associated with Farber lipogranulomatosis; b-hexosaminidase associated with GM2 gangliosidosis and Tay-Sachs and Sandhoff diseases; aspartylglucosaminidase associated with aspartyl-glucosaminuria; α-fucosidase associated with fucosidosis; α-mannosidase associated with alpha-mannosidosis; porphobilinogen deaminase, associated with acute intermittent porphyria (AIP); alpha-1 antitrypsin for treatment of alpha-1 antitrypsin deficiency (emphysema); erythropoietin for treatment of anemia due to thalassemia or to renal failure; vascular endothelial growth factor, angiopoietin-1, and fibroblast growth factor for the treatment of ischemic diseases; thrombomodulin and tissue factor pathway inhibitor for the treatment of occluded blood vessels as seen in, for example, atherosclerosis, thrombosis, or embolisms; aromatic amino acid decarboxylase (AADC), and tyrosine hydroxylase (TH) for the treatment of Parkinson's disease; the beta adrenergic receptor; anti-sense to, or a mutant form of, phospholamban, the sarco(endo)plasmic reticulum adenosine triphosphatase-2 (SERCA2), and the cardiac adenylyl cyclase for the treatment of congestive heart failure; a tumor suppressor gene such as p53 for the treatment of various cancers; a cytokine such as one of the various interleukins for the treatment of inflammatory and immune disorders and cancers; dystrophin or minidystrophin and utrophin or miniutrophin for the treatment of muscular dystrophies; and, insulin or GLP-1 for the treatment of diabetes.

In another embodiment, the transgene comprises more than one transgene. This may be accomplished using a single vector carrying two or more heterologous sequences, or using two or more AAV each carrying one or more heterologous sequences. In one embodiment, the AAV is used for gene suppression (or knockdown) and gene augmentation co-therapy. In knockdown/augmentation co-therapy, the defective copy of the gene of interest is silenced and a non-mutated copy is supplied. In one embodiment, this is accomplished using two or more co-administered vectors. See, Millington-Ward et al, Molecular Therapy, April 2011, 19(4):642-649 which is incorporated herein by reference. The transgenes may be readily selected by one of skill in the art based on the desired result.

### Viral and Non-Viral Vectors

The expression cassette described herein, containing a weak promoter and heterologous coding sequence, may be engineered into any suitable genetic element for delivery to a target cell, such as a vector. A “vector” as used herein is a biological or chemical moiety comprising a nucleic acid sequence which can be introduced into an appropriate host cell for replication or expression of said nucleic acid sequence. Common vectors include non-viral vectors and viral vectors. As used herein, a non-viral system might be selected from nanoparticles, electroporation systems and novel biomaterials, naked DNA, phage, transposon, plasmids, cosmids (Phillip McClean, www.ndsu.edu/pubweb/˜mcclean/-plsc731/cloning/cloning4.htm) and artificial chromosomes (Gong, Shiaoching, et al. “A gene expression atlas of the central nervous system based on bacterial artificial chromosomes.” Nature 425.6961 (2003): 917-925).

“Plasmid” or “plasmid vector” generally is designated herein by a lower case p preceded and/or followed by a vector name. Plasmids, other cloning and expression vectors, properties thereof, and constructing/manipulating methods thereof that can be used in accordance with the present invention are readily apparent to those of skill in the art. In one embodiment, the nucleic acid sequence as described herein or the expression cassette as described herein are engineered into a suitable genetic element (a vector) useful for generating viral vectors and/or for delivery to a host cell, e.g., naked DNA, phage, transposon, cosmid, episome, etc., which transfers the nuclease sequences carried thereon. The selected vector may be delivered by any suitable method, including transfection, electroporation, liposome delivery, membrane fusion techniques, high velocity DNA-coated pellets, viral infection and protoplast fusion. The methods used to make such constructs are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y.

In certain embodiments, the expression cassette is located in a vector genome for packaging into a viral capsid. For example, for an AAV vector genome, the components of the expression cassette are flanked at the extreme 5′ end and the extreme 3′ end by AAV inverted terminal repeat sequences. For example, a 5′ AAV ITR, expression cassette, 3′ AAV ITR. In other embodiments, a self-complementary AAV may be selected. In other embodiments, retroviral system, lentivirus vector system, or an adenoviral system may be used. In one embodiment, the vector genome is that shown in any of SEQ ID NO: 9-14. In one embodiment, the vector genome is that shown in SEQ ID NO: 9. In one embodiment, the vector genome is that shown in SEQ ID NO: 10. In one embodiment, the vector genome is that shown in SEQ ID NO: 11. In one embodiment, the vector genome is that shown in SEQ ID NO: 12. In one embodiment, the vector genome is that shown in SEQ ID NO: 13. In one embodiment, the vector genome is that shown in SEQ ID NO: 14.

### AAV Vectors

In certain embodiments, a recombinant AAV is provided. A “recombinant AAV” or “rAAV” is a DNAse-resistant viral particle containing two elements, an AAV capsid and a vector genome containing at least non-AAV coding sequence packaged within the AAV capsid. Unless otherwise specified, this term may be used interchangeably with the phrase “rAAV vector”. The rAAV is a “replication-defective virus” or “viral vector”, as it lacks any functional AAV rep gene or functional AAV cap gene and cannot generate progeny. In certain embodiments, the only AAV sequences are the AAV inverted terminal repeat sequences (ITRs), typically located at the extreme 5′ and 3′ ends of the vector genome in order to allow the gene and regulatory sequences located between the ITRs to be packaged within the AAV capsid.

The source of the AAV capsid may be one of any of the dozens of naturally occurring and available adeno-associated viruses, as well as engineered AAVs. An adeno-associated virus (AAV) viral vector is an AAV DNase-resistant particle having an AAV protein capsid into which is packaged nucleic acid sequences for delivery to target cells. An AAV capsid is composed of 60 capsid (cap) protein subunits, VP1, VP2, and VP3, that are arranged in an icosahedral symmetry in a ratio of approximately 1:1:10 to 1:1:20, depending upon the selected AAV. Various AAVs may be selected as sources for capsids of AAV viral vectors as identified above. See, e.g., US Published Patent Application No. 2007-0036760-A1; US Published Patent Application No. 2009-0197338-A1; EP 1310571. See also, WO 2003/042397 (AAV7 and other simian AAV), U.S. Pat. Nos. 7,790,449 and 7,282,199 (AAV8), WO 2005/033321 and U.S. Pat. No. 7,906,111 (AAV9), and WO 2006/110689, WO 2003/042397 (rh.10) and WO 2018/160582 (AAVhu68). These documents also describe other AAV which may be selected for generating AAV and are incorporated by reference. Unless otherwise specified, the AAV capsid, ITRs, and other selected AAV components described herein, may be readily selected from among any AAV, including, without limitation, the AAVs commonly identified as AAV1, AAV2, AAV3, AAV4, AAV5, AAV6, AAV7, AAV8, AAV9, AAV8 bp, AAV7M8, AAVAnc80, AAVrh10, and AAVPHP.B and variants of any of the known or mentioned AAVs or AAVs yet to be discovered or variants or mixtures thereof. See, e.g., WO 2005/033321, which is incorporated herein by reference. In one embodiment, the AAV capsid is an AAV1 capsid or variant thereof, AAV8 capsid or variant thereof, an AAV9 capsid or variant thereof, an AAVrh.10 capsid or variant thereof, an AAVrh64R1 capsid or variant thereof, an AAVhu.37 capsid or variant thereof, or an AAV3B or variant thereof. In one aspect, the capsid is an AAVhu.37 capsid. See, also WO 2019/168961 and WO 2019/168961, which are incorporated by reference herein in their entirety.

In other embodiments, the AAV capsid is an AAVrh.79 capsid or variant thereof. In other embodiments, the AAV capsid is an AAVrh.90 or variant thereof.

In certain embodiments, the rAAV comprises an AAVhu37 capsid. An AAVhu37 capsid comprises: a heterogeneous population of vp1 proteins which are the product of a nucleic acid sequence encoding the amino acid sequence of SEQ ID NO: 22, a heterogeneous population of vp2 proteins which are the product of a nucleic acid sequence encoding the amino acid sequence of at least about amino acids 138 to 738 of SEQ ID NO: 22, and a heterogeneous population of vp3 proteins which are the product of a nucleic acid sequence encoding at least amino acids 204 to 738 of SEQ ID NO: 22 wherein: the vp1, vp2 and vp3 proteins contain subpopulations with amino acid modifications comprising at least two highly deamidated asparagines (N) in asparagine-glycine pairs in SEQ ID NO: 22 and optionally further comprising subpopulations comprising other deamidated amino acids, wherein the deamidation results in an amino acid change. AAVhu37 is characterized by having highly deamidated residues, e.g., at positions N57, N263, N385, and/or N514 based on the numbering of the AAVhu37 VP1 (SEQ ID NO: 22).

Deamidation has been observed in other residues, as shown in the table below, and in, e.g., WO 2019/168961, published Sep. 6, 2019, which is incorporated herein by reference. In certain embodiments, an AAVhu37 capsid is modified in one or more of the following positions, in the ranges provided below, as determined using mass spectrometry with a trypsin enzyme. In certain embodiments, one or more of the following positions, or the glycine following the N is modified as described herein. For example, in certain embodiments, a G may be modified to an S or an A, e.g., at position 58, 264, 386, or 515. In one embodiment, the AAVhu37 capsid is modified at position N57/G58 to N57Q or G58A to afford a capsid with reduced deamidation at this position. In another embodiment, N57/G58 is altered to NS57/58 or NA57/58. However, in certain embodiments, an increase in deamidation is observed when NG is altered to NS or NA. In certain embodiments, an N of an NG pair is modified to a Q while retaining the G. In certain embodiments, both amino acids of an NG pair are modified. In certain embodiments, N385Q results in significant reduction of deamidation in that location. In certain embodiments, N499Q results in significant increase of deamidation in that location.

In certain embodiments, AAVhu37 may have these or other residues deamidated, e.g., typically at less than 10% and/or may have other modifications, including methylations (e.g, —R487) (typically less than 5%, more typically less than 1% at a given residue), isomerization (e.g., at D97) (typically less than 5%, more typically less than 1% at a given residue, phosphorylation (e.g., where present, in the range of about 10 to about 60%, or about 10 to about 30%, or about 20 to about 60%) (e.g., at one or more of S149, ˜S153, ˜S474, ˜T570, ˜S665), or oxidation (e.g, at one or more of W248, W307, W307, M405, M437, M473, W480, W480, W505, M526, M544, M561, W621, M637, and/or W697). Optionally the W may oxidize to kynurenine.

TABLE A

| AAVhu37 Deamidation | |
|---|---|
| based on VP1 numbering | % Deamidation |
| N57 + Deamidation | 65-90, 70-95, 80-95, |
| | 75-100, 80-100, or 90-100 |
| N94 + Deamidation | 5-15, about 10 |
| ~N254 + Deamidation | 10-20 |
| ~N263 + Deamidation | 75-100 |
| ~N305 + Deamidation | 1-5 |
| ~N385 + Deamidation | 65-90, 70-95, 80-95, |
| | 75-100, 80-100, or 90-100 |
| ~N410 + Deamidation | 1-25, |
| N479 + Deamidation | 1-5, 1-3 |
| ~N514 + Deamidation | 65-90, 70-95, 80-95, |
| | 75-100, 80-100, or 90-100 |
| ~Q601 + Deamidation | 0-1 |
| N653 + Deamidation | 0-2 |

Still other positions may have such these or other modifications (e.g., acetylation or further deamidations). In certain embodiments, the nucleic acid sequence encoding the AAVhu37 vp1 capsid protein is provided in SEQ ID NO: 21. In other embodiments, a nucleic acid sequence of 70% to 99.9% identity to SEQ ID NO: 21 may be selected to express the AAVhu37 capsid proteins. In certain other embodiments, the nucleic acid sequence is at least about 75% identical, at least 80% identical, at least 85%, at least 90%, at least 95%, at least 97% identical, or at least 99% identical to SEQ ID NO: 21. However, other nucleic acid sequences which encode the amino acid sequence of SEQ ID NO: 22 may be selected for use in producing rAAVhu37 capsids. In certain embodiments, the nucleic acid sequence has the nucleic acid sequence of SEQ ID NO: 21 or a sequence at least 70% to at least 99% identical, at least 75%, at least 80%, at least 85%, at least 90%, at least 95%, at least 97%, at least 99%, identical to SEQ ID NO: 21 which encodes SEQ ID NO: 22. In certain embodiments, the nucleic acid sequence has the nucleic acid sequence of SEQ ID NO: 21 or a sequence at least 70% to 99%, at least 75%, at least 80%, at least 85%, at least 90%, at least 95%, at least 97%, at least 99%, identical to about nt 412 to about nt 2214 of SEQ ID NO: 21 which encodes the vp2 capsid protein (about aa 138 to 738) of SEQ ID NO: 22. In certain embodiments, the nucleic acid sequence has the nucleic acid sequence of about nt 610 to about nt 2214 of SEQ ID NO: 21 or a sequence at least 70% to 99%, at least 75%, at least 80%, at least 85%, at least 90%, at least 95%, at least 97%, at least 99%, identical to nt SEQ ID NO: 21 which encodes the vp3 capsid protein (about aa 204 to 738) of SEQ ID NO: 22. See, EP 2 345 731 B1 and SEQ ID NO: 88 therein, which are incorporated by reference.

In certain embodiments, the rAAV comprises an AAV8 capsid. An AAV8 capsid comprises: a heterogeneous population of VP isoforms which are deamidated as defined in the following table, based on the total amount of VP proteins in the capsid, as determined using mass spectrometry. Suitable modifications include those described in the paragraph above labelled modulation of deamidation, which is incorporated herein. In certain embodiments, the AAV capsid is modified at one or more of the following position, in the ranges provided below, as determined using mass spectrometry. In certain embodiments, one or more of the following positions, or the glycine following the N is modified as described herein. In certain embodiments, an artificial NG is introduced into a different position than one of the positions identified below. In certain embodiments, one or more of the following positions, or the glycine following the N is modified as described herein. For example, in certain embodiments, a G may be modified to an S or an A, e.g., at position 58, 67, 95, 216, 264, 386, 411, 460, 500, 515, or 541. Significant reduction in deamidation is observed when NG57/58 is altered to NS 57/58 or NA57/58. However, in certain embodiments, an increase in deamidation is observed when NG is altered to NS or NA. In certain embodiments, an N of an NG pair is modified to a Q while retaining the G. In certain embodiments, both amino acids of an NG pair are modified. In certain embodiments, N385Q results in significant reduction of deamidation in that location. In certain embodiments, N499Q results in significant increase of deamidation in that location. In certain embodiments, an NG mutation is made at the pair located at N263 (e.g., to N263A). In certain embodiments, an NG mutation is made at the pair located at N514 (e.g., to N514A). In certain embodiments, an NG mutation is made at the pair located at N540 (e.g., N540A). In certain embodiments, AAV mutants containing multiple mutations and at least one of the mutations at these positions are engineered. In certain embodiments, no mutation is made at position N57. In certain embodiments, no mutation is made at position N94. In certain embodiments, no mutation is made at position N305. In certain embodiments, no mutation is made at position G386. In certain embodiments, no mutation is made at position Q467. In certain embodiments, no mutation is made at position N479. In certain embodiments, no mutation is made at position N653. In certain embodiments, the capsid is modified to reduce “N” or “Q” at positions other than then “NG” pairs. Residue numbers are based on the published AAV8 sequence, reproduced in SEQ ID NO: 20.

TABLE B

| AAV8 Modification | |
|---|---|
| Based on VP1 numbering | % |
| N35 + Deamidation | 1 |
| N57 + Deamidation | 65-90, 70-95, 80-95, |
| | 75-100, 80-100, or 90-100 |
| N66 + Deamidation | 0-10 |
| N94 + Deamidation | 1-15 |
| N113 + Deamidation | 0-10 |
| ~Q166 + Deamidation | 0-10 |
| ~N173 + Deamidation | 0-10 |
| N254/N255 + Deamidation | 5-45 |
| N263 + Deamidation | 65-90, 70-95, 80-95, |
| | 75-100, 80-100, or 90-100 |
| ~N304 + Deamidation | 0-10 |
| ~N305 + Deamidation | 10-40 |
| N320 + Deamidation | 0-10 |
| ~Q322 + Deamidation | 0-10 |
| N385 + Deamidation | 65-90, 70-95, 80-95, |
| | 75-100, 80-100, or 90-100 |
| N410 + Deamidation | 15-70 |
| ~Q431 + Deamidation | 0-10 |
| N438 + Deamidation | 0-10 |
| ~N459 + Deamidation | 0-10 |
| ~Q467 + Deamidation | 0-10 |
| ~N479 + Deamidation | 0-10 |
| N498/N499 + Deamidation | 0-10 |
| N502 + Deamidation | 0-10 |
| N514 + Deamidation | 65-90, 70-95, 80-95, |
| | 75-100, 80-100, or 90-100 |
| N517 + Deamidation | 15-40 |
| N540 + Deamidation | 65-90, 70-95, 80-95, |
| | 75-100, 80-100, or 90-100 |
| ~N554 + Deamidation | 0-10 |
| ~Q589 + Deamidation | 0-10 |
| ~N590 + Deamidation | 0-10 |
| ~N599 + Deamidation | 35-75 |
| ~Q601 + Deamidation | 45-75 |
| ~Q610 + Deamidation | 0-10 |
| Q617 + Deamidation | 0-10 |
| N630 + Deamidation | 5-30 |
| Q648 + Deamidation | 0-10 |
| N653 + Deamidation | 0-10 |
| N665 + Deamidation | 5-30 |
| N670 + Deamidation | 0-10 |
| N693 + Deamidation | 0-10 |
| ~N706 + Deamidation | 0-10 |
| N718 + Deamidation | 0-10 |
| N737 + Deamidation | 0-10 |

In certain embodiments, the rAAV comprises a AAVrh79 capsid, as described in WO 2019/169004, published Sep. 6, 2019, which is incorporated herein by reference. In one embodiment, an AAVrh79 capsid comprises a heterogeneous population of AAVrh79 vp1 proteins, AAVrh79 vp2 proteins, and AAVrh79 vp3 proteins. In one embodiment, the AAVrh79 capsid is produced by expression from a nucleic acid sequence which encodes the predicted amino acid sequence of 1 to 738 of SEQ ID NO: 18. Optionally, sequences co-expressing the vp3 protein from a nucleic acid sequence excluding the vp1-unique region (about aa 1 to 137) or the vp2-unique region (about aa 1 to 203), vp1 proteins produced from SEQ ID NO: 17, or vp1 proteins produced from a nucleic acid sequence at least 70% identical to SEQ ID NO: 17 which encodes the predicted amino acid sequence of 1 to 738 of SEQ ID NO: 18. In other embodiments, the AAVrh79 vp2 proteins produced by expression from a nucleic acid sequence which encodes the predicted amino acid sequence of at least about amino acids 138 to 738 of SEQ ID NO: 18, vp2 proteins produced from a sequence comprising at least nucleotides 412 to 2214 of SEQ ID NO: 17, or vp2 proteins produced from a nucleic acid sequence at least 70% identical to at least nucleotides 412 to 2214 of SEQ ID NO: 18 which encodes the predicted amino acid sequence of at least about amino acids 138 to 738 of SEQ ID NO: 18, AAVrh79 vp3 proteins produced by expression from a nucleic acid sequence which encodes the predicted amino acid sequence of at least about amino acids 204 to 738 of SEQ ID NO: 18, vp3 proteins produced from a sequence comprising at least nucleotides 610 to 2214 of SEQ ID NO: 17, or vp3 proteins produced from a nucleic acid sequence at least 70% identical to at least nucleotides 610 to 2214 of SEQ ID NO: 17 which encodes the predicted amino acid sequence of at least about amino acids 204 to 738 of SEQ ID NO: 18.

In certain embodiments, an AAVrh79 capsid comprises: a heterogeneous population of vp1 proteins which are the product of a nucleic acid sequence encoding the amino acid sequence of SEQ ID NO: 18, a heterogeneous population of vp2 proteins which are the product of a nucleic acid sequence encoding the amino acid sequence of at least about amino acids 138 to 738 of SEQ ID NO: 18, and a heterogeneous population of vp3 proteins which are the product of a nucleic acid sequence encoding at least amino acids 204 to 738 of SEQ ID NO: 18.

The AAVrh79 vp1, vp2 and vp3 proteins contain subpopulations with amino acid modifications comprising at least two highly deamidated asparagines (N) in asparagine-glycine pairs in SEQ ID NO: 18 and optionally further comprising subpopulations comprising other deamidated amino acids, wherein the deamidation results in an amino acid change. High levels of deamidation at N-G pairs N57, N263, N385 and/or N514 are observed, relative to the number of SEQ ID NO: 18. Deamidation has been observed in other residues, as shown in the table below and in the examples. In certain embodiments, AAVrh79 may have other residues deamidated, e.g., typically at less than 10% and/or may have other modifications, including methylations (e.g, ˜R487) (typically less than 5%, more typically less than 1% at a given residue), isomerization (e.g., at D97) (typically less than 5%, more typically less than 1% at a given residue, phosphorylation (e.g., where present, in the range of about 10 to about 60%, or about 10 to about 30%, or about 20 to about 60%) (e.g., at one or more of S149, ˜S153, ˜S474, ˜T570, ˜S665), or oxidation (e.g, at one or more of W248, W307, W307, M405, M437, M473, W480, W480, W505, M526, M544, M561, W621, M637, and/or W697). Optionally the W may oxidize to kynurenine.

TABLE C
AAVrh79 Deamidation

| AAVrh79 Deamidation | |
|---|---|
| based on VP1 numbering | % Deamidation |
| N57 + Deamidation | 65-90, 70-95, 80-95, |
| | 75-100, 80-100, or 90-100 |
| N94 + Deamidation | 5-15, about 10 |
| ~N254 + Deamidation | 10-20 |
| ~N263 + Deamidation | 75-100 |
| ~N305 + Deamidation | 1-5 |
| ~N385 + Deamidation | 65-90, 70-95, 80-95, |
| | 75-100, 80-100, or 90-100 |
| ~N410 + Deamidation | 1-25, |
| N479 + Deamidation | 1-5, 1-3 |
| ~N514 + Deamidation | 65-90, 70-95, 80-95, |
| | 75-100, 80-100, or 90-100 |
| ~Q601 + Deamidation | 0-1 |
| N653 + Deamidation | 0-2 |

In certain embodiments, an AAVrh79 capsid is modified in one or more of the positions identified in the preceding table, in the ranges provided below, as determined using mass spectrometry with a trypsin enzyme. In certain embodiments, one or more of the following positions, or the glycine following the N is modified as described herein. Residue numbers are based on the AAVrh79 sequence provided herein. See, SEQ ID NO: 18.

In certain embodiments, the nucleic acid sequence encoding the AAVrh79 vp1 capsid protein is provided in SEQ ID NO: 17. In other embodiments, a nucleic acid sequence of 70% to 99.9% identity to SEQ ID NO: 17 may be selected to express the AAVrh79 capsid proteins. In certain other embodiments, the nucleic acid sequence is at least about 75% identical, at least 80% identical, at least 85%, at least 90%, at least 95%, at least 97% identical, at least 99% or at least 99.9% identical to SEQ ID NO: 17. However, other nucleic acid sequences which encode the amino acid sequence of SEQ ID NO: 18 may be selected for use in producing rAAV capsids. In certain embodiments, the nucleic acid sequence has the nucleic acid sequence of SEQ ID NO: 17 or a sequence at least 70% to 99% identical, at least 75%, at least 80%, at least 85%, at least 90%, at least 95%, at least 97%, at least 99%, or at least 99.9% identical to SEQ ID NO: 17 which encodes SEQ ID NO: 18. In certain embodiments, the nucleic acid sequence has the nucleic acid sequence of SEQ ID NO: 17 or a sequence at least 70% to 99.%, at least 75%, at least 80%, at least 85%, at least 90%, at least 95%, at least 97%, at least 99%, or at least 99.9% identical to about nt 412 to about nt 2214 of SEQ ID NO: 17 which encodes the vp2 capsid protein (about aa 138 to 738) of SEQ ID NO: 18. In certain embodiments, the nucleic acid sequence has the nucleic acid sequence of about nt 610 to about nt 2214 of SEQ ID NO: 17 or a sequence at least 70% to 99.%, at least 75%, at least 80%, at least 85%, at least 90%, at least 95%, at least 97%, at least 99%, or at least 99.9% identical to nt SEQ ID NO: 17 which encodes the vp3 capsid protein (about aa 204 to 738) of SEQ ID NO: 18.

The invention also encompasses nucleic acid sequences encoding mutant AAVrh79, in which one or more residues has been altered in order to decrease deamidation, or other modifications which are identified herein. Such nucleic acid sequences can be used in production of mutant rAAVrh79 capsids.

In certain embodiments, the rAAV comprises a AAVrh.90 capsid, as described in WO 2020/223232, published Nov. 5, 2020, which is incorporated herein by reference. In a further aspect, a recombinant adeno-associated virus (rAAV) is provided which comprises: (A) an AAVrh.90 capsid comprising one or more of: (1) AAVrh.90 capsid proteins comprising: a heterogeneous population of AAVrh.90 vp1 proteins selected from: vp1 proteins produced by expression from a nucleic acid sequence which encodes the predicted amino acid sequence of 1 to 738 of SEQ ID NO: 24, vp1 proteins produced from SEQ ID NO: 23, or vp1 proteins produced from a nucleic acid sequence at least 70% identical to SEQ ID NO: 23 which encodes the predicted amino acid sequence of 1 to 738 of SEQ ID NO: 24, a heterogeneous population of AAVrh.90 vp2 proteins selected from: vp2 proteins produced by expression from a nucleic acid sequence which encodes the predicted amino acid sequence of at least about amino acids 138 to 738 of SEQ ID NO: 24, vp2 proteins produced from a sequence comprising at least nucleotides 412 to 2214 of SEQ ID NO: 23, or vp2 proteins produced from a nucleic acid sequence at least 70% identical to at least nucleotides 412 to 2214 of SEQ ID NO: 23 which encodes the predicted amino acid sequence of at least about amino acids 138 to 738 of SEQ ID NO: 24, a heterogeneous population of AAVrh.90 vp3 proteins selected from: vp3 proteins produced by expression from a nucleic acid sequence which encodes the predicted amino acid sequence of at least about amino acids 204 to 738 of SEQ ID NO: 24, vp3 proteins produced from a sequence comprising at least nucleotides 610 to 2214 of SEQ ID NO: 23, or vp3 proteins produced from a nucleic acid sequence at least 70% identical to at least nucleotides 610 to 2214 of SEQ ID NO: 23 which encodes the predicted amino acid sequence of at least about amino acids 204 to 738 of SEQ ID NO: 24; and/or (2) a heterogeneous population of vp1 proteins which are the product of a nucleic acid sequence encoding the amino acid sequence of SEQ ID NO: 24, a heterogeneous population of vp2 proteins which are the product of a nucleic acid sequence encoding the amino acid sequence of at least about amino acids 138 to 738 of SEQ ID NO: 24, and a heterogeneous population of vp3 proteins which are the product of a nucleic acid sequence encoding at least amino acids 204 to 738 of SEQ ID NO: 24, wherein: the vp1, vp2 and vp3 proteins contain subpopulations with amino acid modifications comprising at least two highly deamidated asparagines (N) in asparagine-glycine pairs in SEQ ID NO: 24 and optionally further comprising subpopulations comprising other deamidated amino acids, wherein the deamidation results in an amino acid change; and (B) a vector genome in the AAVrh.90 capsid, the vector genome comprising a nucleic acid molecule comprising AAV inverted terminal repeat sequences and a non-AAV nucleic acid sequence encoding a product operably linked to sequences which direct expression of the product in a host cell.

In certain embodiments, the AAVrh.90 vp1, vp2 and vp3 proteins contain subpopulations with amino acid modifications comprising at least two highly deamidated asparagines (N) in asparagine-glycine pairs in SEQ ID NO: 24 and optionally further comprising subpopulations comprising other deamidated amino acids, wherein the deamidation results in an amino acid change. High levels of deamidation at N-G pairs N57, ˜N263, ˜N385, and/or ˜N514 are observed, relative to the number of SEQ ID NO: 24. Deamidation has been observed in other residues as shown in the table below. In certain embodiments, AAVrh.90 may have other residues deamidated (e.g., ˜N305, ˜N499, and/or ˜N599, typically at less than 20%) and/or may have other modifications, including phosphorylation (e.g., where present, in the range of about 2 to about 30%, or about 2 to about 20%, or about 2 to about 10%) (e.g., at S149), or oxidation (e.g, at one or more of ˜W23, ˜M204, ˜M212, W248, W282, M405, M473, W480, W505, M526, ˜N544, M561, and/or ˜M607). Optionally the W may oxidize to kynurenine.

TABLE D
AAVrh.90 Deamidation

| AAVrh.90 Deamidation | |
|---|---|
| based on VP1 numbering | % Deamidation |
| N57 + Deamidation | 65-90, 70-95, 80-95, |
| | 75-100, 80-100, or 90-100 |
| N94 + Deamidation | 2-15 or 2-5 |
| ~N263 + Deamidation | 65-90, 70-95, 80-95, |
| | 75-100, 80-100, or 90-100 |
| ~N305 + Deamidation | 5-30, 5-20, or 10-20 |
| ~N385 + Deamidation | 65-90, 70-95, 80-95, |
| | 75-100, 80-100, or 90-100 |
| ~N499 + Deamidation | 2-15, 2-10, or 5-10 |
| ~N514 + Deamidation | 65-90, 70-95, 80-95, |
| | 75-100, 80-100, or 90-100 |
| ~N599 + Deamidation | 2-15, 2-10, or 5-10 |

In certain embodiments, an AAVrh.90 capsid is modified in one or more of the positions identified in the preceding table, in the ranges provided, as determined using mass spectrometry with a trypsin enzyme. In certain embodiments, one or more of the positions, or the glycine following the N is modified as described herein. Residue numbers are based on the AAVrh.90 sequence provided herein. See, SEQ ID NO: 24.

In certain embodiments, an AAVrh.90 capsid comprises: a heterogeneous population of vp1 proteins which are the product of a nucleic acid sequence encoding the amino acid sequence of SEQ ID NO: 24, a heterogeneous population of vp2 proteins which are the product of a nucleic acid sequence encoding the amino acid sequence of at least about amino acids 138 to 738 of SEQ ID NO: 24, and a heterogeneous population of vp3 proteins which are the product of a nucleic acid sequence encoding at least amino acids 204 to 738 of SEQ ID NO: 24.

As used herein, a “vector genome” refers to the nucleic acid sequence packaged inside the rAAV capsid which forms a viral particle. Such a nucleic acid sequence contains AAV inverted terminal repeat sequences (ITRs). In the examples herein, a vector genome contains, at a minimum, from 5′ to 3′, an AAV 5′ ITR, expression cassette containing the transgene or coding sequence(s) operably linked to regulatory sequences directing expression thereof, and an AAV 3′ ITR. The ITRs are the genetic elements responsible for the replication and packaging of the genome during vector production and are the only viral cis elements required to generate rAAV. In one embodiment, the ITRs are from an AAV different than that supplying a capsid. In a preferred embodiment, the ITR sequences from AAV2, or the deleted version thereof (ΔITR), which may be used for convenience. However, ITRs from other AAV sources may be selected. Where the source of the ITRs is from AAV2 and the AAV capsid is from another AAV source, the resulting vector may be termed pseudotyped. Typically, AAV vector genome comprises an AAV 5′ ITR, the nucleic acid sequences encoding the gene product(s) and any regulatory sequences, and an AAV 3′ ITR. However, other configurations of these elements may be suitable. In one embodiment, a self-complementary AAV is provided. A shortened version of the 5′ ITR, termed ΔITR, has been described in which the D-sequence and terminal resolution site (trs) are deleted. In certain embodiments, the vector genome includes a shortened AAV2 ITR of 130 base pairs, wherein the external “a” element is deleted. The shortened ITR is reverted back to the wild-type length of 145 base pairs during vector DNA amplification using the internal A element as a template. In other embodiments, the full-length AAV 5′ and 3′ ITRs are used. In other embodiments, a full-length or engineered ITR may be selected. Further, the vector genome contains regulatory sequences that modulate expression of the gene products (e.g, directly or indirectly by modulating transcription and/or translation). Suitable components of a vector genome are discussed in more detail herein.

For use in producing an AAV viral vector (e.g., a recombinant (r) AAV), the expression cassettes can be carried on any suitable vector, e.g., a plasmid, which is delivered to a packaging host cell. The plasmids useful in this invention may be engineered such that they are suitable for replication and packaging in vitro in prokaryotic cells, insect cells, mammalian cells, among others. Suitable transfection techniques and packaging host cells are known and/or can be readily designed by one of skill in the art. In one embodiment, the vector genome shown in SEQ ID NO: 13 is packaged into an AAVhu.37 capsid.

Methods for generating and isolating AAVs suitable for use as vectors are known in the art. See generally, e.g., Grieger & Samulski, 2005, “Adeno-associated virus as a gene therapy vector: Vector development, production and clinical applications,” Adv. Biochem. Engin/Biotechnol. 99: 119-145; Buning et al., 2008, “Recent developments in adeno-associated virus vector technology,” J. Gene Med. 10:717-733; and the references cited below, each of which is incorporated herein by reference in its entirety. For packaging a transgene into virions, the ITRs are the only AAV components required in cis in the same construct as the nucleic acid molecule containing the expression cassettes. The cap and rep genes can be supplied in trans.

The term “AAV intermediate” or “AAV vector intermediate” refers to an assembled rAAV capsid which lacks the desired genomic sequences packaged therein. These may also be termed an “empty” capsid. Such a capsid may contain no detectable genomic sequences of an expression cassette, or only partially packaged genomic sequences which are insufficient to achieve expression of the gene product. These empty capsids are non-functional to transfer the gene of interest to a host cell.

The recombinant adeno-associated virus (AAV) described herein may be generated using techniques which are known. See, e.g., WO 2003/042397; WO 2005/033321, WO 2006/110689; U.S. Pat. No. 7,588,772 B2. Such a method involves culturing a host cell which contains a nucleic acid sequence encoding an AAV capsid protein; a functional rep gene; an expression cassette composed of, at a minimum, AAV inverted terminal repeats (ITRs) and a transgene; and sufficient helper functions to permit packaging of the expression cassette into the AAV capsid protein. Methods of generating the capsid, coding sequences therefor, and methods for production of rAAV viral vectors have been described. See, e.g., Gao, et al, Proc. Natl. Acad. Sci. U.S.A. 100 (10), 6081-6086 (2003) and US 2013/0045186A1.

In one embodiment, a production cell culture useful for producing a recombinant AAV is provided. Such a cell culture contains a nucleic acid which expresses the AAV capsid protein in the host cell; a nucleic acid molecule suitable for packaging into the AAV capsid, e.g., a vector genome which contains AAV ITRs and a non-AAV nucleic acid sequence encoding a gene product operably linked to sequences which direct expression of the product in a host cell; and sufficient AAV rep functions and adenovirus helper functions to permit packaging of the nucleic acid molecule into the recombinant AAV capsid. In one embodiment, the cell culture is composed of mammalian cells (e.g., human embryonic kidney 293 cells, among others) or insect cells (e.g., baculovirus).

Optionally the rep functions are provided by an AAV other than the AAV providing the capsid. For example the rep may be, but is not limited to, AAV1 rep protein, AAV2 rep protein, AAV3 rep protein, AAV4 rep protein, AAV5 rep protein, AAV6 rep protein, AAV7 rep protein, AAV8 rep protein; or rep 78, rep 68, rep 52, rep 40, rep68/78 and rep40/52; or a fragment thereof; or another source. Optionally, the rep and cap sequences are on the same genetic element in the cell culture. There may be a spacer between the rep sequence and cap gene. Any of these AAV or mutant AAV capsid sequences may be under the control of exogenous regulatory control sequences which direct expression thereof in a host cell.

In one embodiment, cells are manufactured in a suitable cell culture (e.g., HEK 293) cells. Methods for manufacturing the gene therapy vectors described herein include methods well known in the art such as generation of plasmid DNA used for production of the gene therapy vectors, generation of the vectors, and purification of the vectors. In some embodiments, the gene therapy vector is an AAV vector and the plasmids generated are an AAV cis-plasmid encoding the AAV genome and the gene of interest, an AAV trans-plasmid containing AAV rep and cap genes, and an adenovirus helper plasmid. The vector generation process can include method steps such as initiation of cell culture, passage of cells, seeding of cells, transfection of cells with the plasmid DNA, post-transfection medium exchange to serum free medium, and the harvest of vector-containing cells and culture media. The harvested vector-containing cells and culture media are referred to herein as crude cell harvest. In yet another system, the gene therapy vectors are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, the contents of each of which is incorporated herein by reference in its entirety. Methods of making and using these and other AAV production systems are also described in the following U.S. patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065.

The crude cell harvest may thereafter be subject method steps such as concentration of the vector harvest, diafiltration of the vector harvest, microfluidization of the vector harvest, nuclease digestion of the vector harvest, filtration of microfluidized intermediate, crude purification by chromatography, crude purification by ultracentrifugation, buffer exchange by tangential flow filtration, and/or formulation and filtration to prepare bulk vector.

A two-step affinity chromatography purification at high salt concentration followed anion exchange resin chromatography are used to purify the vector drug product and to remove empty capsids. These methods are described in more detail in International Patent Publication No. WO 2017/160360, which is incorporated by reference herein. Purification methods for AAV8, International Patent Publication No. WO 2017/100676, and rh10, International Patent Publication No. WO 2017/100704, and for AAV1, International Patent Publication No. WO 2017/100674 are all incorporated by reference herein.

To calculate empty and full particle content, VP3 band volumes for a selected sample (e.g., in examples herein an iodixanol gradient-purified preparation where # of GC=#of particles) are plotted against GC particles loaded. The resulting linear equation (y=mx+c) is used to calculate the number of particles in the band volumes of the test article peaks. The number of particles (pt) per 20 μL loaded is then multiplied by 50 to give particles (pt)/mL. Pt/mL divided by GC/mL gives the ratio of particles to genome copies (pt/GC). Pt/mL-GC/mL gives empty pt/mL. Empty pt/mL divided by pt/mL and x 100 gives the percentage of empty particles.

Generally, methods for assaying for empty capsids and AAV vector particles with packaged genomes have been known in the art. See, e.g., Grimm et al., Gene Therapy (1999) 6:1322-1330; Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J. Virol. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using SilverXpress (Invitrogen, CA) according to the manufacturer's instructions or other suitable staining method, i.e. SYPRO ruby or coomassie stains. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

In one aspect, an optimized q-PCR method is used which utilizes a broad spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2-fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000 fold) and subjected to TaqMan analysis as described in the standard assay.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum Gene Ther Methods. 2014 April; 25(2):115-25. doi: 10.1089/hgtb.2013.131. Epub 2014 Feb. 14.

In brief, the method for separating rAAV particles having packaged genomic sequences from genome-deficient AAV intermediates involves subjecting a suspension comprising recombinant AAV viral particles and AAV capsid intermediates to fast performance liquid chromatography, wherein the AAV viral particles and AAV intermediates are bound to a strong anion exchange resin equilibrated at a high pH, and subjected to a salt gradient while monitoring eluate for ultraviolet absorbance at about 260 and about 280. The pH may be adjusted depending upon the AAV selected. See, e.g., WO2017/160360 (AAV9), WO2017/100704 (AAVrh10), WO 2017/100676 (e.g., AAV8), and WO 2017/100674 (AAV1)] which are incorporated by reference herein. In this method, the AAV full capsids are collected from a fraction which is eluted when the ratio of A260/A280 reaches an inflection point. In one example, for the Affinity Chromatography step, the diafiltered product may be applied to a Capture Select™ Poros-AAV2/9 affinity resin (Life Technologies) that efficiently captures the AAV2 serotype. Under these ionic conditions, a significant percentage of residual cellular DNA and proteins flow through the column, while AAV particles are efficiently captured.

### Pharmaceutical Compositions

A pharmaceutical composition comprises one or more of an expression cassette, vector containing same (viral or non-viral) or another system containing the expression cassette and one or more of a carrier, suspending agent, and/or excipient.

In certain embodiments, compositions containing at least one rAAV stock (e.g., an rAAV stock) and an optional carrier, excipient and/or preservative. An rAAV stock refers to a plurality of rAAV vectors which are the same, e.g., such as in the amounts described below in the discussion of concentrations and dosage units.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host. Delivery vehicles such as liposomes, nanocapsules, microparticles, microspheres, lipid particles, vesicles, and the like, may be used for the introduction of the compositions of the present invention into suitable host cells. In particular, the rAAV vector delivered vector genomes may be formulated for delivery either encapsulated in a lipid particle, a liposome, a vesicle, a nanosphere, or a nanoparticle or the like.

In certain embodiments, an expression cassette is delivered via a lipid nanoparticle. The term “lipid nanoparticle” refers to a lipid composition having a typically spherical structure with an average diameter of 10 to 1000 nanometers, e.g. 75 nm to 750 nm, or 100 nm and 350 nm, or between 250 nm to about 500 nm. In some formulations, lipid nanoparticles can comprise at least one cationic lipid, at least one noncationic lipid, and at least one conjugated lipid. Lipid nanoparticles known in the art that are suitable for encapsulating nucleic acids, such as mRNA, may be used. “Average diameter” is the average size of the population of nanoparticles comprising the lipophilic phase and the hydrophilic phase. The mean size of these systems can be measured by standard methods known by the person skilled in the art. Examples of suitable lipid nanoparticles for gene therapy is described, e.g., L. Battaglia and E. Ugazio, J Nanomaterials, Vol 2019, Article ID 283441, pp. 1-22; US2012/0183589A1; and WO 2012/170930 which are incorporated herein by reference in their entirety.

In one embodiment, a composition includes a final formulation suitable for delivery to a subject, e.g., is an aqueous liquid suspension buffered to a physiologically compatible pH and salt concentration. Optionally, one or more surfactants are present in the formulation. In another embodiment, the composition may be transported as a concentrate which is diluted for administration to a subject. In other embodiments, the composition may be lyophilized and reconstituted at the time of administration.

Methods and agents well known in the art for making formulations are described, for example, in “Remington's Pharmaceutical Sciences,” Mack Publishing Company, Easton, Pa. Formulations may, for example, contain excipients, carriers, stabilizers, or diluents such as sterile water, saline, polyalkylene glycols such as polyethylene glycol, oils of vegetable origin, or hydrogenated napthalenes, preservatives (such as octadecyldimethylbenzyl, ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, and m-cresol), low molecular weight polypeptides, proteins such as serum albumin, gelatin, or immunoglobulins, hydrophilic polymers such as polyvinylpyrrolidone, amino acids such as glycine, glutamine, asparagine, histidine, arginine, and lysine, monosaccharides, disaccharides, and other carbohydrates including glucose, mannose, and dextrins, chelating agents such as EDTA, sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions. Such techniques are disclosed in Remington's Pharmaceutical Sciences 16th edition, Osol, A. Ed. (1980).

A suitable surfactant, or combination of surfactants, may be selected from among non-ionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension.

The vectors are administered in sufficient amounts to transfect the cells and to provide sufficient levels of gene transfer and expression to provide a therapeutic benefit without undue adverse effects, or with medically acceptable physiological effects, which can be determined by those skilled in the medical arts. Conventional and pharmaceutically acceptable routes of administration include, but are not limited to, direct delivery to a desired organ (e.g., the liver (optionally via the hepatic artery), lung, heart, eye, kidney,), oral, inhalation, intranasal, intrathecal, intratracheal, intraarterial, intraocular, intravenous, intramuscular, subcutaneous, intradermal, and other parental routes of administration. Routes of administration may be combined, if desired.

Dosages of the viral vector depend primarily on factors such as the condition being treated, the age, weight and health of the patient, and may thus vary among patients. For example, a therapeutically effective human dosage of the viral vector is generally in the range of from about 25 to about 1000 microliters to about 100 mL of solution containing concentrations of from about 1×10⁹ to 1×10¹⁶ genomes virus vector. The dosage is adjusted to balance the therapeutic benefit against any side effects and such dosages may vary depending upon the therapeutic application for which the recombinant vector is employed. The levels of expression of the transgene product can be monitored to determine the frequency of dosage resulting in viral vectors, preferably AAV vectors containing the minigene. Optionally, dosage regimens similar to those described for therapeutic purposes may be utilized for immunization using the compositions of the invention.

The replication-defective virus compositions can be formulated in dosage units to contain an amount of replication-defective virus that is in the range of about 1.0×10⁹ GC to about 1.0×10¹⁶ GC (to treat an average subject of 70 kg in body weight) including all integers or fractional amounts within the range, and preferably 1.0×10¹² GC to 1.0×10¹⁴ GC for a human patient. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per dose including all integers or fractional amounts within the range. In another embodiment, the compositions are formulated to contain at least 1×10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 1×10¹², 2×10¹², 3×10¹², 4×10¹², 5×10¹², 6×10¹², 7×10¹², 8×10¹², or 9×10¹² GC per dose including all integers or fractional amounts within the range. In another embodiment, the compositions are formulated to contain at least 1×10¹³, 2×10¹³, 3×10¹³, 4×10¹³, 5×10¹³, 6×10¹³, 7×10¹³, 8×10¹³, or 9×10¹³ GC per dose including all integers or fractional amounts within the range. In another embodiment, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per dose including all integers or fractional amounts within the range. In another embodiment, the compositions are formulated to contain at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range. In one embodiment, for human application the dose can range from 1×10¹⁰ to about 1×10¹² GC per dose including all integers or fractional amounts within the range.

These above doses may be administered in a variety of volumes of carrier, excipient or buffer formulation, ranging from about 25 to about 1000 microliters, or higher volumes, including all numbers within the range, depending on the size of the area to be treated, the viral titer used, the route of administration, and the desired effect of the method.

Any suitable route of administration may be selected. Accordingly, pharmaceutical compositions may be formulated for any appropriate route of administration, for example, in the form of liquid solutions or suspensions (as, for example, for intravenous administration, for oral administration, etc.). Alternatively, pharmaceutical compositions may be in solid form (e.g., in the form of tablets or capsules, for example for oral administration). In some embodiments, pharmaceutical compositions may be in the form of powders, drops, aerosols, etc.

### Methods

The compositions provided herein are useful for reducing off-target activity of enzymes delivered in vivo. In certain embodiments, the compositions are useful in reducing off-target activity of an enzyme expressed following non-viral mediated delivery of an expression cassette comprising the enzyme coding sequence under the control of a weak promoter, as described herein. In certain embodiments, the compositions are useful in reducing off-target activity of an enzyme expressed following AAV-mediated delivery of a vector genome.

In one embodiment, a method for editing a targeted gene is provided. The method includes delivering a nuclease expression cassette comprising a nucleic acid comprising a nuclease coding sequence which is operably linked to regulatory sequences which direct expression of the nuclease following delivery to a host cell having a sequence to which the nuclease is targeted, wherein the regulatory sequences comprise a promoter which has low transcriptional activity. Such promoters are described herein. In another embodiment, the method includes delivering a composition, viral vector or rAAV comprising the expression cassette, as described herein.

In another embodiment, a method for reducing off-target activity of a gene targeting nuclease is provided. The method includes delivering a nuclease expression cassette comprising a nucleic acid comprising a nuclease coding sequence which is operably linked to regulatory sequences which direct expression of the nuclease following delivery to a host cell having a sequence to which the nuclease is targeted, wherein the regulatory sequences comprise a promoter which has low transcriptional activity. Such promoters are described herein. In another embodiment, the method includes delivering a composition, viral vector or rAAV comprising the expression cassette, as described herein.

In certain embodiments, the effectiveness of a weak promoter may be assessed in vitro. For example, the half-life of a nuclease may be assessed in vitro (in cultured cells) by treating the cells to stop translation of the protein (e.g., with cycloheximide (CHX)) and then performing a western blot at different times post-treatment. Other suitable methods for assessing off-targeting activity of a nuclease may be readily determined by one of skill in the art.

A reduction in off-target nuclease activity can be determined using a variety of approaches which have been described in the literature. Such methods for determining nuclease specificity, include cell-free methods such as Site-Seq [Cameron, P., et al, (2017) Mapping the genomic landscape of CRISPR-Cas9 cleavage. Nat Methods, 14, 600-606], Digenome-seq [Kim, D., et al, (2015) Digenome-seq: genome-wide profiling of CRISPR-Cas9 off-target effects in human cells. Nat Methods, 12, 237-243, 231 p following 243], and Circle-Seq [Tsai, S. Q., et al, (2017) CIRCLE-seq: a highly sensitive in vitro screen for genome-wide CRISPR-Cas9 nuclease off-targets. Nat Methods, 14, 607-614] and in vitro-based methods such as, e.g., GUIDE-Seq [Tsai (2017) Nat Methods, 14, 607-614] and Integrative-Deficient Lentiviral Vectors Capture (IDLV) [Gabriel, R., et al. (2011) An unbiased genome-wide analysis of zinc-finger nuclease specificity. Nat Biotechnol, 29, 816-823; Wang, X., et al., (2015) Unbiased detection of off-target cleavage by CRISPR-Cas9 and TALENs using integrase-defective lentiviral vectors. Nat Biotechnol, 33, 175-178].

In some embodiments, the off-target activity is assessed by ITR-seq. See, e.g., the publication Breton et al, ITR-Seq, a next-generation sequencing assay, identifies genome-wide DNA editing sites in vivo following adeno-associated viral vector-mediated genome editing, BMC Genomics, (2020):21:239 which is incorporated herein by reference in its entirety.

In one aspect, a method for editing a targeted gene is provided which comprises delivering a nuclease expression cassette under control of a weak promoter as described herein.

In one aspect, a method for editing a targeted gene is provided which comprises delivering a composition as described herein.

In one aspect, a method for editing a targeted gene is provided which comprises delivering a viral or non-viral vector as described herein.

In one aspect, a method for editing a targeted gene is provided which comprises delivering an rAAV as described herein.

In one aspect, a method for treating a patient having a cholesterol-related disorder(s), such as hypercholesterolemia, using a nuclease expression cassette comprising a meganuclease which recognizes a site within the human PCSK9 gene, under the control of a weak promoter as described herein. In one embodiment, the weak promoter is F64. In another embodiment, the weak promoter is F113. In another embodiment, the weak promoter is F140. In yet another embodiment, the weak promoter is a CCL16 promoter. In yet another embodiment, the weak promoter is a SCLC22A9 promoter. In yet another embodiment, the weak promoter is a CYP26A1 promoter. Such expression cassettes may be delivered via a viral or non-viral vector. In certain embodiments, the expression cassettes may be delivered using an LNP.

In one aspect, provided is a method for treating a patient having a disorder associated with a defect in the alanine glyoxylate aminotransferase gene, such as primary hyperoxaluria type 1, using a nuclease expression cassette comprising a meganuclease which recognizes a site within the human HAO gene, under the control of a weak promoter as described herein. In one embodiment, the weak promoter is F64. In another embodiment, the weak promoter is F113. In another embodiment, the weak promoter is F140. In yet another embodiment, the weak promoter is a CCL16 promoter. In yet another embodiment, the weak promoter is a SCLC22A9 promoter. In yet another embodiment, the weak promoter is a CYP26A1 promoter. Such expression cassettes may be delivered via a viral or non-viral vector. In certain embodiments, the expression cassettes may be delivered using an LNP. In certain embodiments, the disorder is primary hyperoxaluria (PH1).

In one aspect, a method for treating a patient having a disorder associated with a defect in the transthyretin (TTR) gene is provided, using a nuclease expression cassette comprising a meganuclease which recognizes a site within the human TTR gene under control of a weak promoter as described herein. In one embodiment, the weak promoter is F64. In another embodiment, the weak promoter is F113. In another embodiment, the weak promoter is F140. In yet another embodiment, the weak promoter is a CCL16 promoter. In yet another embodiment, the weak promoter is a SCLC22A9 promoter. In yet another embodiment, the weak promoter is a CYP26A1 promoter. Such expression cassettes may be delivered via a viral or non-viral vector. In certain embodiments, the expression cassettes may be delivered using an LNP. In certain embodiments, the disorder is TTR-related hereditary amyloidosis.

In another aspect, a method for treating a patient having a disorder associated with a defect in the apoliprotein C-II (APOC3) gene is provided, using a nuclease expression cassette comprising a meganuclease which recognizes a site within the human APOC3 gene, under the control of a weak promoter as described herein. In one embodiment, the weak promoter is F64. In another embodiment, the weak promoter is F113. In another embodiment, the weak promoter is F140. In yet another embodiment, the weak promoter is a CCL16 promoter. In yet another embodiment, the weak promoter is a SCLC22A9 promoter. In yet another embodiment, the weak promoter is a CYP26A1 promoter. Such expression cassettes may be delivered via a viral or non-viral vector. In certain embodiments, the expression cassettes may be delivered using an LNP.

In one aspect, a method for treating a patient having a disorder associated with a defect in the branched-chain a-ketoacid dehydrogenase complex (BCKDC) Ela gene is provided, using a nuclease expression cassette comprising a meganuclease which recognizes a site within the human BCKDC Ela gene, under the control of a weak promoter as described herein. In one embodiment, the weak promoter is F64. In another embodiment, the weak promoter is F113. In another embodiment, the weak promoter is F140. In yet another embodiment, the weak promoter is a CCL16 promoter. In yet another embodiment, the weak promoter is a SCLC22A9 promoter. In yet another embodiment, the weak promoter is a CYP26A1 promoter. Such expression cassettes may be delivered via a viral or non-viral vector. In certain embodiments, the expression cassettes may be delivered using an LNP. In certain embodiments, the disorder is maple syrup urine disease.

In one aspect, a method for editing a gene, using a CRISPR/Cas-associated nuclease is provided, using an expression cassette comprising a coding sequence for a CRISPR/Cas-associated nuclease which recognizes a site within the desired gene, under the control of a weak promoter as described herein. In one embodiment, the weak promoter is F64. In another embodiment, the weak promoter is F113. In another embodiment, the weak promoter is F140. In yet another embodiment, the weak promoter is a CCL16 promoter. In yet another embodiment, the weak promoter is a SCLC22A9 promoter. In yet another embodiment, the weak promoter is a CYP26A1 promoter. Such expression cassettes may be delivered via a viral or non-viral vector. In certain embodiments, the expression cassettes may be delivered using an LNP.

In one aspect, a method for editing a gene, using a TALEN is provided, using an expression cassette comprising a TALEN coding sequence which recognizes a site within the desired gene, under the control of a weak promoter as described herein. In one embodiment, the weak promoter is F64. In another embodiment, the weak promoter is F113. In another embodiment, the weak promoter is F140. In yet another embodiment, the weak promoter is a CCL16 promoter. In yet another embodiment, the weak promoter is a SCLC22A9 promoter. In yet another embodiment, the weak promoter is a CYP26A1 promoter. Such expression cassettes may be delivered via a viral or non-viral vector. In certain embodiments, the expression cassettes may be delivered using an LNP.

In one aspect, a method for editing a gene, using a zinc finger nuclease is provided, using an expression cassette comprising a coding sequence for a zinc finger nuclease which recognizes a site within the desired gene, under the control of a weak promoter as described herein. In one embodiment, the weak promoter is F64. In another embodiment, the weak promoter is F113. In another embodiment, the weak promoter is F140. In yet another embodiment, the weak promoter is a CCL16 promoter. In yet another embodiment, the weak promoter is a SCLC22A9 promoter. In yet another embodiment, the weak promoter is a CYP26A1 promoter. Such expression cassettes may be delivered via a viral or non-viral vector. In certain embodiments, the expression cassettes may be delivered using an LNP.

In one aspect, a method for editing a gene, using a meganuclease is provided, using an expression cassette comprising a coding sequence for a meganuclease which recognizes a site within the desired gene, under the control of a weak promoter as described herein. In one embodiment, the weak promoter is F64. In another embodiment, the weak promoter is F113. In another embodiment, the weak promoter is F140. In yet another embodiment, the weak promoter is a CCL16 promoter. In yet another embodiment, the weak promoter is a SCLC22A9 promoter. In yet another embodiment, the weak promoter is a CYP26A1 promoter. Such expression cassettes may be delivered via a viral or non-viral vector. In certain embodiments, the expression cassettes may be delivered using an LNP.

In certain embodiments, nucleases other than meganucleases targeting any of the above-described genes are contemplated.

In certain embodiments, a nuclease expression cassette, non-viral vector, viral vector (e.g., rAAV), or any of the same in a pharmaceutical composition, as described herein is administrable for gene editing in a patient. In certain embodiments, the method is useful for non-embryonic gene editing. In certain embodiments, the patient is an infant (e.g., birth to about 9 months). In certain embodiments, the patient is older than an infant, e.g, 12 months or older.

As used herein, “a,” “an,” or “the” can mean one or more than one. For example, “a” cell can mean a single cell or a multiplicity of cells.

In certain embodiments, the term “meganuclease” refers to an endonuclease that binds double-stranded DNA at a recognition sequence that is greater than 12 base pairs. Preferably, the recognition sequence for a meganuclease of the invention is 22 base pairs. A meganuclease can be an endonuclease that is derived from I-CreI, and can refer to an engineered variant of I-CreI that has been modified relative to natural I-CreI with respect to, for example, DNA-binding specificity, DNA cleavage activity, DNA-binding affinity, or dimerization properties. Methods for producing such modified variants of I-CreI are known in the art. See, e.g., WO 2007/047859). A meganuclease as used herein binds to double-stranded DNA as a heterodimer. A meganuclease may also be a “single-chain meganuclease” in which a pair of DNA-binding domains are joined into a single polypeptide using a peptide linker. The term “homing endonuclease” is synonymous with the term “meganuclease.” See, WO 2018/195449, describing certain PCSK9 meganucleases, which is incorporated herein in its entirety. In one embodiment, the meganuclease is not the ARCUS meganuclease described herein.

As used herein, the term “specificity” means the ability of a meganuclease to recognize and cleave double-stranded DNA molecules only at a particular sequence of base pairs referred to as the recognition sequence, or only at a particular set of recognition sequences. The set of recognition sequences will share certain conserved positions or sequence motifs, but may be degenerate at one or more positions. A highly-specific meganuclease is capable of cleaving only one or a very few recognition sequences. Specificity can be determined by any method known in the art.

The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

As used herein, the term “operably linked” refers to both expression control sequences that are contiguous with the gene of interest and expression control sequences that act in trans or at a distance to control the gene of interest.

The term “exogenous” as used to describe a nucleic acid sequence or protein means that the nucleic acid or protein does not naturally occur in the position in which it exists in a chromosome, or host cell. An exogenous nucleic acid sequence also refers to a sequence derived from and inserted into the same expression cassette or host cell, but which is present in a non-natural state, e.g. a different copy number, or under the control of different regulatory elements.

The term “heterologous” when used with reference to a protein or a nucleic acid indicates that the protein or the nucleic acid comprises two or more sequences or subsequences which are not found in the same relationship to each other in nature. For instance, the nucleic acid is typically recombinantly produced, having two or more sequences from unrelated genes arranged to make a new functional nucleic acid. For example, in one embodiment, the nucleic acid has a promoter from one gene arranged to direct the expression of a coding sequence from a different gene.

As used herein, the term “host cell” may refer to the packaging cell line in which a vector (e.g., a recombinant AAV) is produced from a production plasmid. In the alternative, the term “host cell” may refer to any target cell in which expression of the transgene is desired. Thus, a “host cell,” refers to a prokaryotic or eukaryotic cell that contains a exogenous or heterologous nucleic acid sequence that has been introduced into the cell by any means, e.g., electroporation, calcium phosphate precipitation, microinjection, transformation, viral infection, transfection, liposome delivery, membrane fusion techniques, high velocity DNA-coated pellets, viral infection and protoplast fusion. In certain embodiments herein, the term “host cell” refers to cultures of cells of various mammalian species for in vitro assessment of the compositions described herein. In other embodiments herein, the term “host cell” refers to the cells employed to generate and package the viral vector or recombinant virus. Still in other embodiment, the term “host cell” is intended to reference the target cells of the subject being treated in vivo for the diseases or conditions as described herein. In certain embodiments, the term “host cell” is a liver cell or hepatocyte.

A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”—containing only the gene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

The terms “sequence identity” “percent sequence identity” or “percent identical” in the context of nucleic acid sequences refers to the residues in the two sequences which are the same when aligned for maximum correspondence. The length of sequence identity comparison may be over the full-length of the genome, the full-length of a gene coding sequence, or a fragment of at least about 500 to 5000 nucleotides, is desired. However, identity among smaller fragments, e.g. of at least about nine nucleotides, usually at least about 20 to 24 nucleotides, at least about 28 to 32 nucleotides, at least about 36 or more nucleotides, may also be desired. Similarly, “percent sequence identity” may be readily determined for amino acid sequences, over the full-length of a protein, or a fragment thereof. Suitably, a fragment is at least about 8 amino acids in length and may be up to about 700 amino acids. Examples of suitable fragments are described herein.

The term “substantial homology” or “substantial similarity,” when referring to amino acids or fragments thereof, indicates that, when optimally aligned with appropriate amino acid insertions or deletions with another amino acid (or its complementary strand), there is amino acid sequence identity in at least about 95 to 99% of the aligned sequences. Preferably, the homology is over full-length sequence, or a protein thereof, e.g., a cap protein, a rep protein, or a fragment thereof which is at least 8 amino acids, or more desirably, at least 15 amino acids in length. Examples of suitable fragments are described herein.

By the term “highly conserved” is meant at least 80% identity, preferably at least 90% identity, and more preferably, over 97% identity. Identity is readily determined by one of skill in the art by resort to algorithms and computer programs known by those of skill in the art.

Generally, when referring to “identity”, “homology”, or “similarity” between two different adeno-associated viruses, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence. In the examples, AAV alignments are performed using the published AAV9 sequences as a reference point. Alignments are performed using any of a variety of publicly or commercially available Multiple Sequence Alignment Programs. Examples of such programs include, “Clustal Omega”, “Clustal W”, “CAP Sequence Assembly”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference. Multiple sequence alignment programs are also available for amino acid sequences, e.g., the “Clustal Omega”, “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

As used herein, the term “about” refers to a variant of ±10% from the reference integer and values therebetween. For example “about” 40 base pairs, includes ±4 (i.e., 36-44, which includes the integers 36, 37, 38, 39, 40, 41, 42, 43, 44). For other values, particularly when reference is to a percentage (e.g., 90% identity, about 10% variance, or about 36% mismatches), the term “about” is inclusive of all values within the range including both the integer and fractions.

As used throughout this specification and the claims, the terms “comprising”, “containing”, “including”, and its variants are inclusive of other components, elements, integers, steps and the like. Conversely, the term “consisting” and its variants are exclusive of other components, elements, integers, steps and the like.

Unless defined otherwise in this specification, technical and scientific terms used herein have the same meaning as commonly understood by one of ordinary skill in the art and by reference to published texts, which provide one skilled in the art with a general guide to many of the terms used in the present application.

### EXAMPLES

The ARCUS nuclease (I-CreI endonuclease, further engineered by Precision BioSciences) recognizes and cuts a 22 bp target sequence in the DNA. Cellular proteins recognize and repair these breaks in the DNA. A consequence of this repair mechanism is the insertions or deletions (indels) of nucleotides in the edited loci, these modifications will affect the expression of the corresponding gene.

We, and others, have observed a high percentage of editing in the DNA target region, in both mice and rhesus macaques studies, after Adeno Associated Viral (AAV) vectors-mediated delivering of the ARCUS nuclease. However, sequences similar to the on-target region also were shown to contain indels, indicating an off-target activity of the ARCUS nuclease.

We hypothesized that a certain level of M2PCSK9 is needed for the on-target editing and that increasing nuclease expression over this threshold results in off-target activity. To reduce the levels of M2PCSK9 expression, we replaced the parental TBG promoter in the AAV constructs with promoters with low transcriptional activity.

### Example 1—In Vivo Mouse Study

We, and others, have observed a high percentage of editing in the DNA target region, in both mice and rhesus macaques studies, after Adeno Associated Viral (AAV) vectors-mediated delivering of the ARCUS nuclease. However, sequences similar to the on-target region also were shown to contain indels, indicating an off-target activity of the ARCUS nuclease. This off-target activity is undesirable and, specially for clinical studies, it is imperative to reduce or eliminate this off-target activity, while retaining the high on-target efficacy.

Our hypothesis is that, in contrast with gene therapy, where high expression of the transgene is desirable, genome editing might require a lower transgene expression, while higher expression will also promote off-target editing. Therefore, the aim of this invention is to reduce the transgene expression by reducing its transcription. This could be achieved by selecting liver-specific promoters with weak transcriptional activity.

Selection of candidate promoters was done by two methods. In the first approach, we identified liver-specific human genes with low RNA expression. We searched the Human Atlas Protein database, using the Consensus transcript expression levels (NX level) as a parameter of the transcriptional activity and we selected genes whose transcription was also enriched on liver.

The TBG (thyroid hormone-binding globulin) promoter has been shown to be useful for AAV-mediated delivery of transgenes into the liver. We selected three genes with decreasing NX levels that were also enriched in liver. We obtained the promoter region for these genes from SwitchGear Genomics (Carlsbad, Calif.) (Table 1).

| | Full | Promoter | RNA tissue |
|---|---|---|---|
| Name | name | region | specificity |
| SERPINA7 | Serpin family A | chrX(−)106038724- | Tissue |
| (TBG) | member 7 | 106039200 | enriched |
| | | | (liver) |
| CCL16 | C-C motif | chr17(−): | Tissue |
| | chemokine | 35981355-35982332 | enriched |
| | ligand 16 | | (liver) |
| SLC22A9 | Solute carrier | chr11(+): | Tissue |
| | family 22 | 63369189-63370107 | enriched |
| | member 9 | | (liver) |
| CYP26A1 | Cytochrome | chr10(+): | Group |
| | P450 family 26 | 93073083-93073881 | enriched |
| | subfamily A | | (liver, |
| | member 1 | | retina) |

TABLE 2
Weak promoter characteristics

| | Promoter | | Liver | AAV Titer |
|---|---|---|---|---|
| Vector | length (bp) | Intron | expression¹ | (GC/mL) |
| AAV.TBG | 697 | Yes | 101.8 * | 9.9 × 10¹³ |
| AAV.CCL16 | 978 | No | 79.1 | 1.4 × 10¹⁴ |
| AAV.CYP26A1 | 799 | No | 23.2 | 1.9 × 10¹⁴ |
| AAV.SLC22A9 | 919 | No | 32.2 | 1.1 × 10¹⁴ |
| AAV.TBG-S1-F64 | 64 | No | TBD | 2.2 × 10¹⁴ |
| AAV.TBG-S1-F113 | 113 | No | TBD | 2.0 × 10¹⁴ |
| AAV.TBG-S1-F140 | 140 | No | TBD | 1.4 × 10¹⁴ |

1 Consensus normalized expression (NX) from Human Protein Atlas available from http://www.proteinatlas.org. * Data for parental TBG (without enhancers)

For our second approach, we aimed to reduce the transcriptional activity of the TBG-S1 promoter, a smaller (176 bp) version of the TBG promoter, by shortening its sequence. Starting from the upstream region, we remove increasing lengths of this sequence, the resulting promoters TBG-S1-F140 (F140), TBG-S1-F113 (F113), and TBG-S1-F64 (F64), contained 140, 113, or 64 bp of the TBG-S1 promoter respectively.

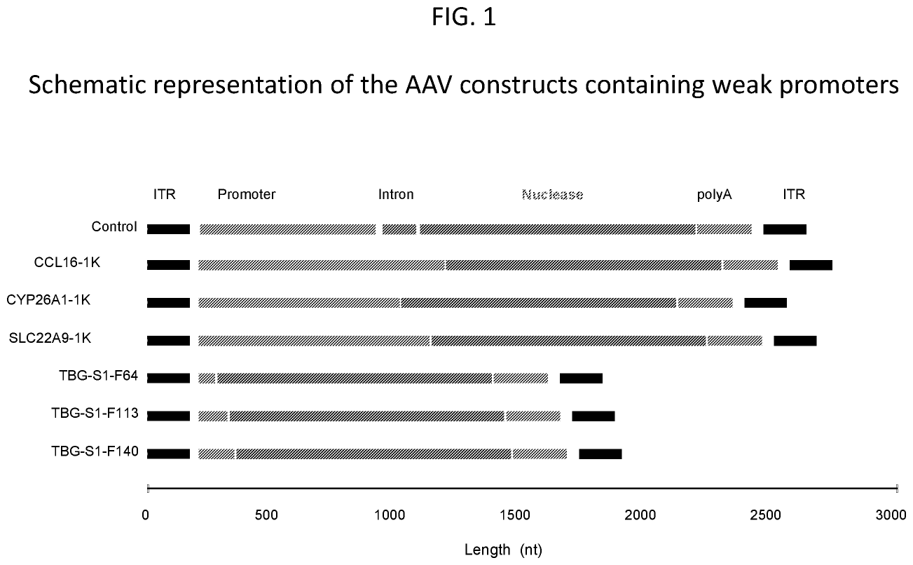

AAV serotype 8 vectors, in which the expression of the ARCUS nuclease, specific for PCSK9, is mediated by one of these six weak promoters, were produced. A schematic representation of the genome of these AAVs is shown in FIG. 1. The following vectors were produced:
a) AAV8.CCL16-1k.ARCUS2.bGH
b) AAV8.CYP26A1-1k.ARCUS2.bGH
c) AAV8.SLC22A9-1k.ARCUS2.bGH
d) AAV8.TBG-S1-F64.ARCUS2.bGH
e) AAV8.TBG-S1-F113.ARCUS2.bGH
f) AAV8.TBG-S1-F140.ARCUS2.bGH

An initial test was performed in mice. Briefly, mice were administered with AAV expressing human PCSK9, two weeks later, mice received a second injection of AAV expressing the PCSK9-specific ARCUS nuclease under the different weak promoters. As a positive control we used a construct in which the nuclease expression is mediated by the TBG promoter. At week 7 post administration of the second vector, mice were euthanized, and liver collected for further analysis.

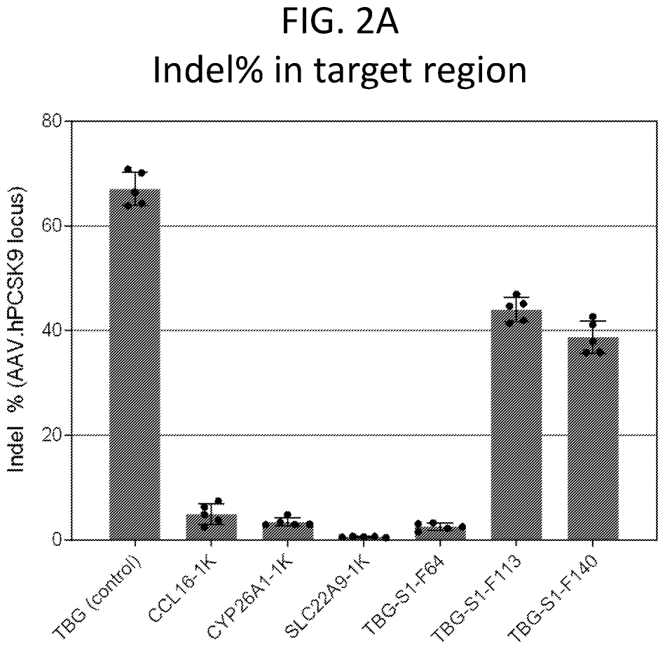

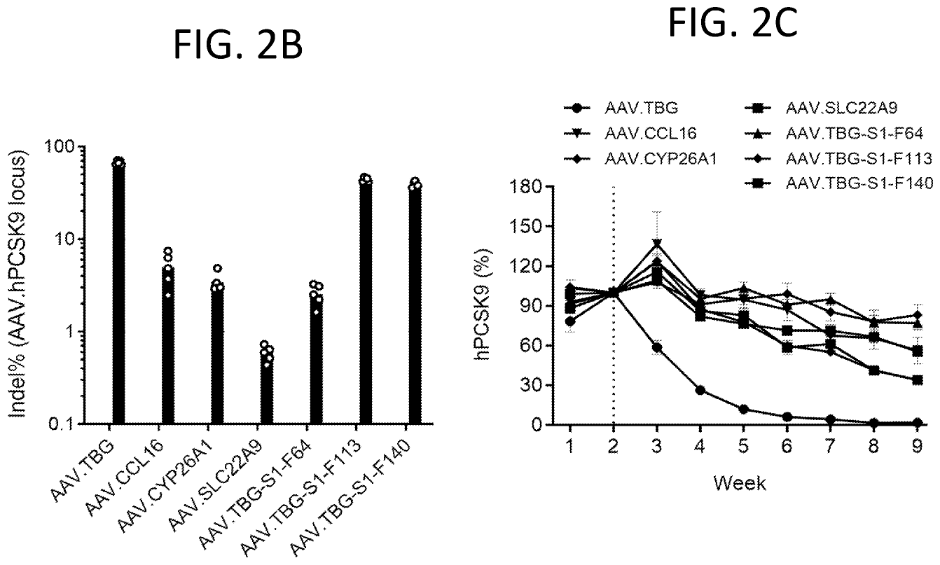

The levels of indels in the region corresponding to the target sequence of the ARCUS nuclease were quantified by a next-generation sequencing assay (FIG. 2A, 2B). The results show that in two of the weak promoters groups (TBG-S1-F113 and TBG-S1-F140) the indel percentage was around 40% at week 7 post-nuclease administration, indicating that the on-target activity is retained. In the rest of the groups the on-target activity was lower than 10%, except for the TBG control group in which the editing was between 60-70% (FIG. 2A, 2B (linear and logarithmic scales, respectively)). FIG. 2C shows Average levels of recombinant PCSK9 in serum, determined by an ELISA assay, per treated group.

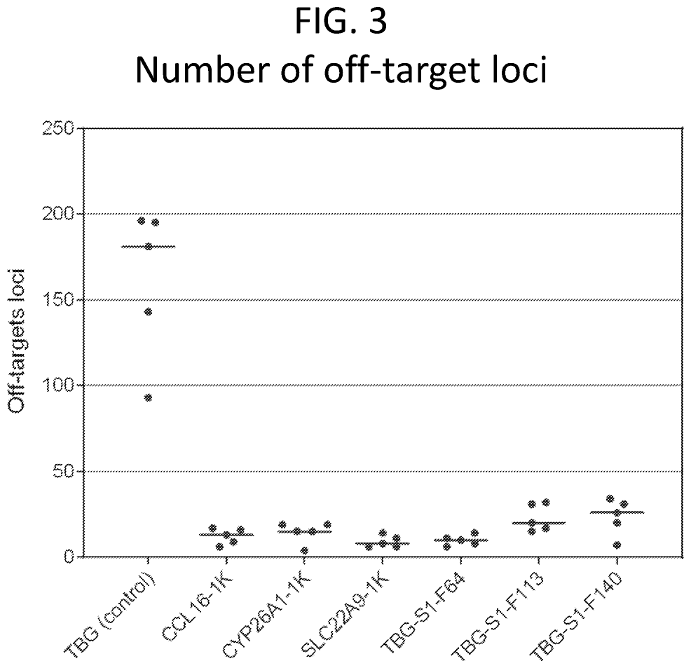

Then, the number of off-target loci in the genomic DNA as a result of the nuclease activity was determined using an NGS-based method called ITR-Seq. The publication Breton et al, ITR-Seq, a next-generation sequencing assay, identifies genome-wide DNA editing sites in vivo following adeno-associated viral vector-mediated genome editing, BMC Genomics, (2020):21:239 is incorporated herein by reference in its entirety. There is a reduction in the number of off-target loci for all the weak promoter groups compared to the TBG control in which the number of off-targets was around 160 (FIG. 3).

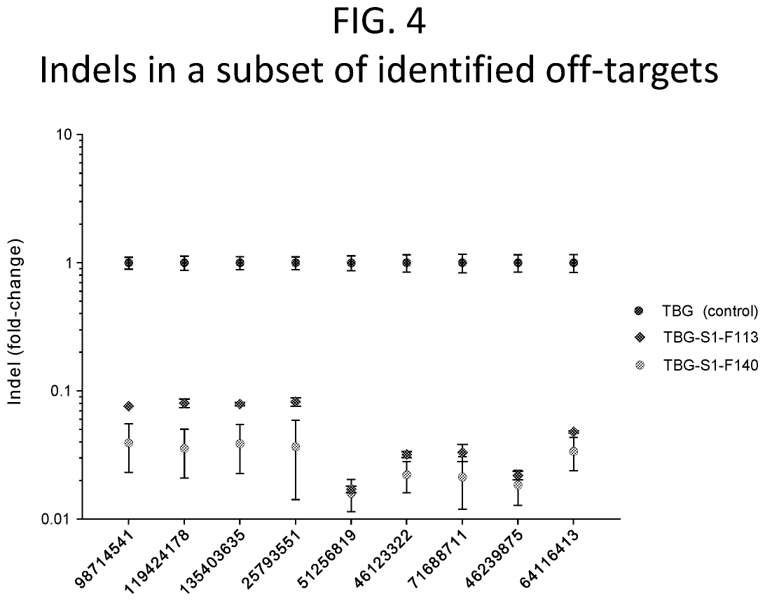

A more quantitative approach to measure the off-target activity of these vectors was to calculate indels in a subset of the identified off-targets. The analysis was only performed in TBG control, TBG-S1-F113 and TBG-S1-F140 groups, as this showed the highest indel % (FIG. 2A, 2B). FIG. 4 shows the indels in a set of genomic locations corresponding to the identified off-targets. Indels levels for each off-target are shown relative to the indels levels in TBG control group (arbitrary value of 1). There was an approximately 20-fold reduction in the indels in the analyzed weak promoters groups, indicating that the use of these promoters clearly reduces the nuclease off-target activity.

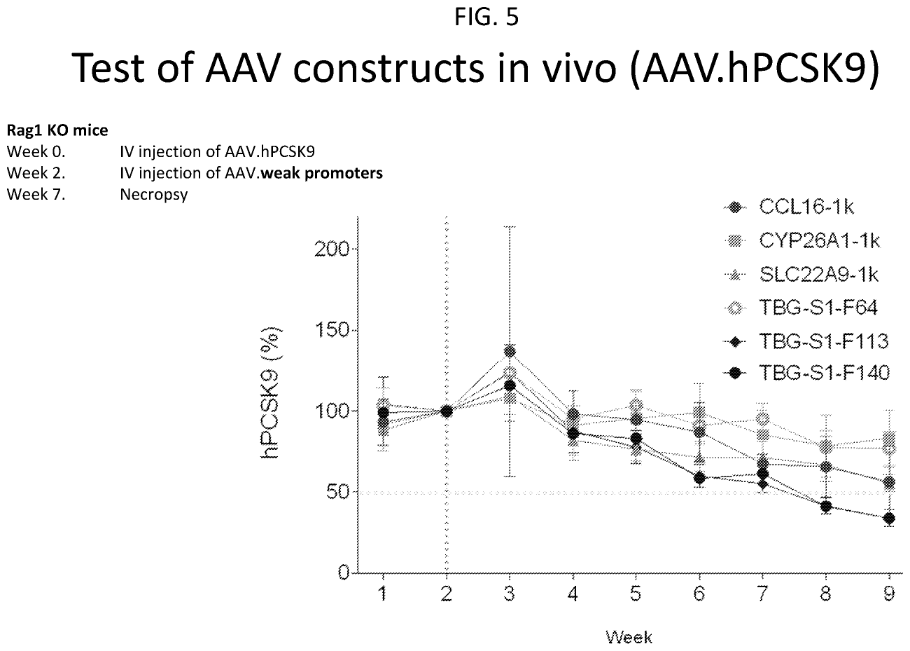

hPCSK9 levels in the injected mice are shown in FIG. 5.

Overall, these results show that the use of weak liver-specific promoters to mediate the expression of genome editing nucleases is a promising strategy to reduce their off-target activity while retaining on-target activity.

### Example 2—NHP Pilot Study

To observe if the reduction in off-targets was conserved in NHP, rhesus macaques were treated with the vectors at a dose of 6×10¹² GC/kg. Biopsy data was collected at d18 (will be followed-up to 1 year). AAV Weekly bleeds were performed until d28 after vector administration, then biweekly until the end of the study. Vectors tested were AAV8.TBG.M2PCSK9 and AAV8.TBG-S1-F113.M2PCSK9. The following studies were performed: Neutralizing antibodies to AAV8 capsid; CBC/Chem/Coag/lipid panel; Serum for PCSK9 expression by ELISA; PBMC isolation every 8 weeks for IFN-g ELISPOT; Liver biopsies at d18 and d128; DNA/RNA analysis to detect on-target and off-target genome editing by next generation sequencing.

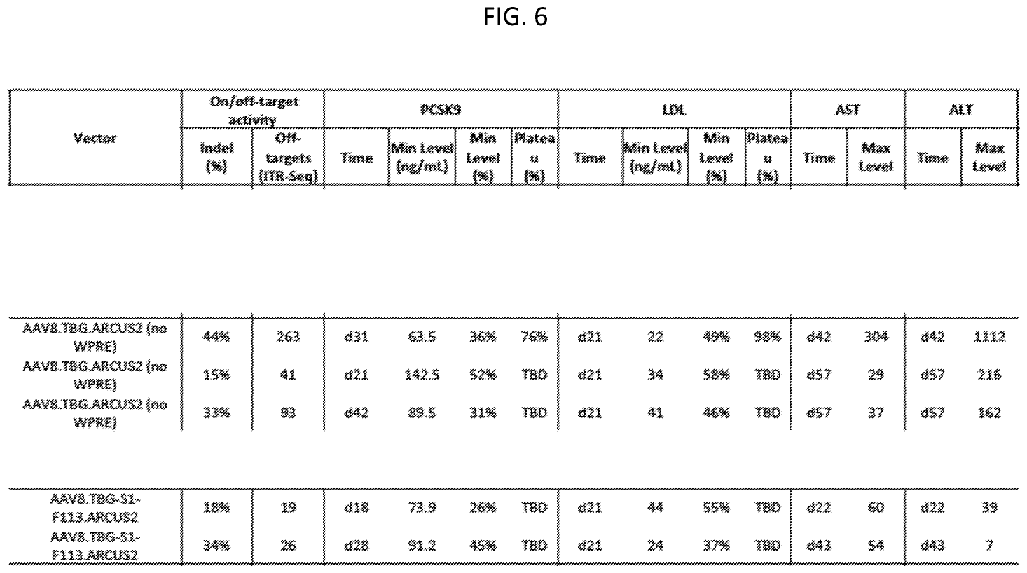

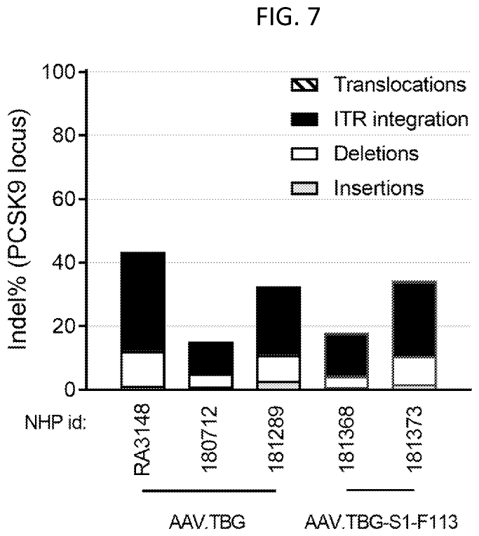

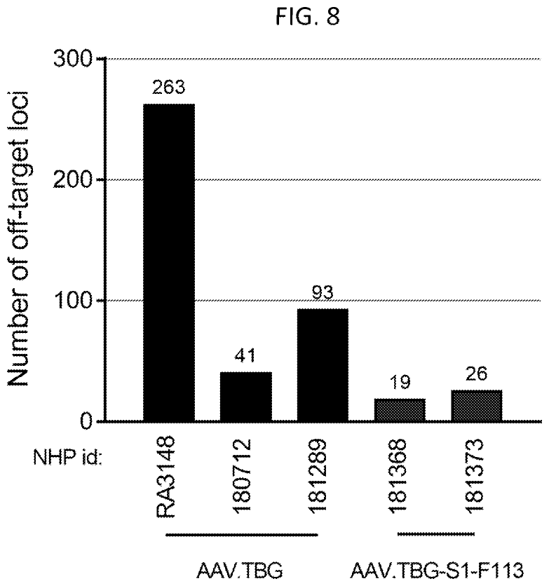

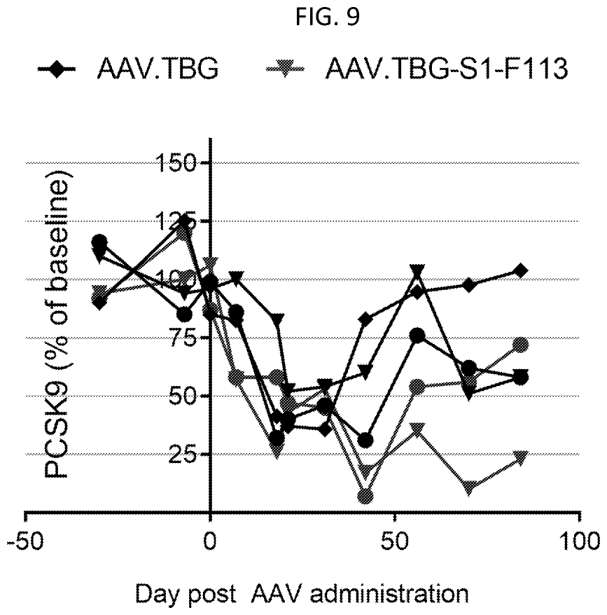

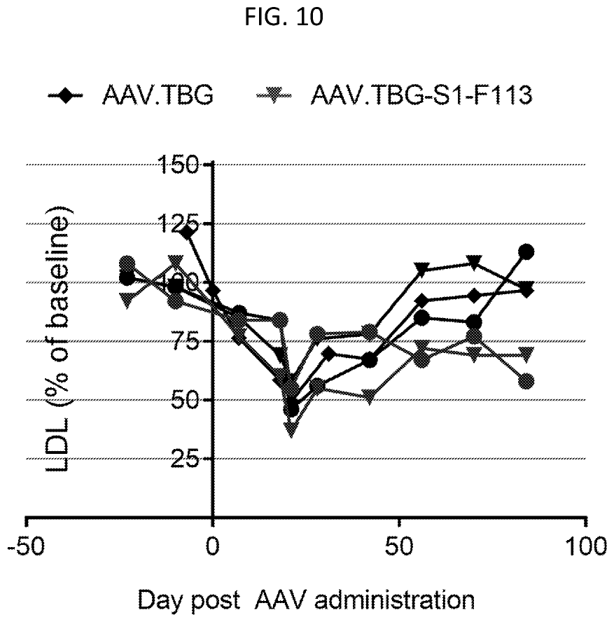

A summary of some of the data presented in FIGS. 7-10 is shown in FIG. 6. Indel % (FIG. 7) and number of off-targets (FIG. 8) were determined in DNA from liver biopsies at day 18 post-AAV. PCSK9 levels (as a percentage of baseline) are shown in FIG. 9 (for 7 weeks post-AAV). LDL levels (as a percentage of baseline) are shown in FIG. 10.

We observed a similar reduction in PCSK9 and LDL levels in the treated NHPs. Expressing the ARCUS nuclease through the use of weak promoters reduces the nuclease off-target activity in mouse and NHP while retaining its on-target activity against the PCSK9 gene.

### Example 3—GLP Toxicity Study

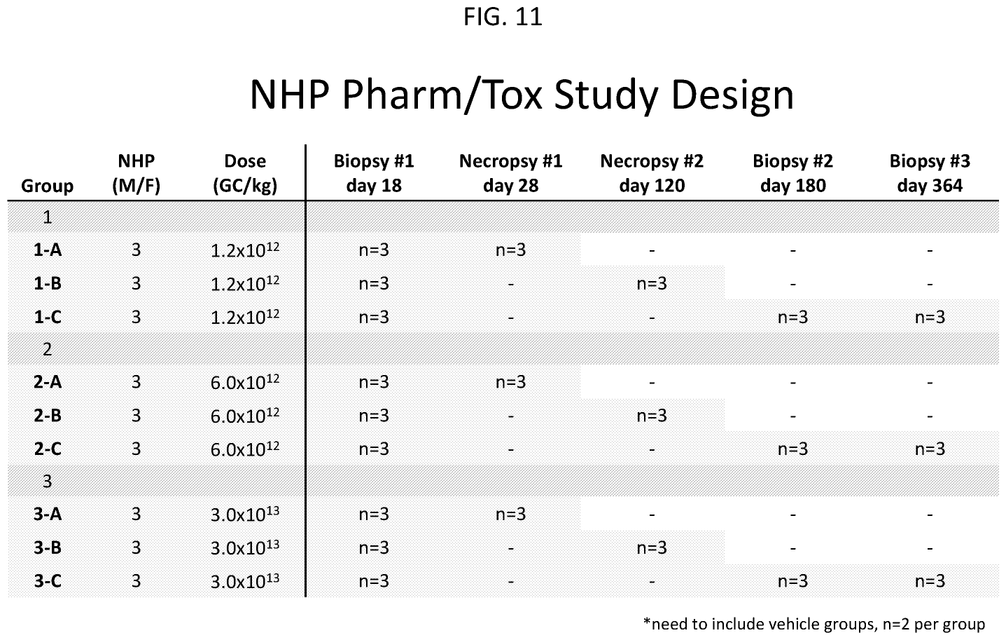

GLP toxicity study is performed in NHPs (n=27) with d120 data and follow-up to 1 y (minimum). The study design is shown in FIG. 11. IV administration of the AAVhu37.TBG-S1-F113.M2PCSK9 vector containing the vector genome shown in SEQ ID NO: 13 is provided at one of three doses: 1.2e12, 6.0e12, 3.0e13. Weekly bleeds are performed until d28 after vector administration, then biweekly until the end of the study. The following studies are performed: Neutralizing antibodies to AAVhu.37 capsid, CBC/Chem/Coag/lipid panel, Serum for PCSK9 expression by ELISA, PBMC isolation every 8 weeks for IFN-g ELISPOT, Liver biopsy at d18 for all NHPs, DNA/RNA analysis to detect on-target and off-target genome editing by next generation sequencing; d28 necropsy for 3 NHP per group—histopathology and biodistribution for all the major organs; d120 necropsy for 3 NHP per group—histopathology and biodistribution for all the major organs; and liver biopsies for the last three NHP per group at d180 and d364.

We expect to observe a similar reduction in PCSK9 and LDL levels in the treated NHPs as in the studies described above. We expect that expressing the ARCUS nuclease through the use of weak promoters will reduce the nuclease off-target activity in NHP while retaining its on-target activity against the PCSK9 gene.

All documents cited in this specification, as well as Breton et al, Increasing the Specificity of AAV-Based Gene Editing through Self-Targeting and Short-Promoter Strategies, Mol Ther. 2021 Mar. 3; 29(3):1047-1056. doi: 10.1016/j.ymthe.2020.12.028. Epub 2020 Dec. 25. are incorporated herein by reference. U.S. Provisional Patent Application No. 63/016,541, filed Apr. 27, 2020, U.S. Provisional Patent Application No. 63/033,738, filed Jun. 2, 2020, U.S. Provisional Patent Application No. 63/089,796, filed Oct. 9, 2020, and 63/016,139, filed Apr. 27, 2020, are incorporated by reference in their entireties, together with their sequence listings. The sequence listing filed herewith named “20-9267PCT_Seq-Listing_ST25.txt” and the sequences and text therein are incorporated by reference. While the invention has been described with reference to particular embodiments, it will be appreciated that modifications can be made without departing from the spirit of the invention. Such modifications are intended to fall within the scope of the appended claims.

| SEQ ID NO | Free text |
|---|---|
| 1 | <213> Artificial Sequence |
| | <223> CRE motif |
| 2 | <213> Artificial Sequence |
| | <223> CRE Recognition sequence |
| 6-8 | <213> Artificial Sequence |
| | <223> constructed sequence |
| 9 | <213> Artificial Sequence |
| | <223> constructed sequence |
| | <221> misc_feature |
| | <222> (1) . . . (168) |
| | <223> ITR |
| | <221> misc_feature |
| | <222> (206) . . . (1183) |
| | <223> CCL16 promoter |
| | <221> misc_feature |
| | <222> (1193) . . . (2287) |
| | <223> PCS7-8L.197 CDS |
| | <221> misc_feature |
| | <222> (2298) . . . (2512) |
| | <223> PolyA |
| | <221> misc_feature |
| | <222> (2562) . . . (2729) |
| | <223> ITR |
| 10 | <213> Artificial Sequence |
| | <223> constructed sequence |
| | <221> misc_feature |
| | <222> (1) . . . (168) |
| | <223> ITR |
| | <221> misc_feature |
| | <222> (206) . . . (1124) |
| | <223> SLC22A9 promoter |
| | <221> misc_feature |
| | <222> (1133) . . . (2227) |
| | <223> PCS7-8L CDS |
| | <221> misc_feature |
| | <222> (2238) . . . (2452) |
| | <223> BGH Poly A |
| | <221> misc_feature |
| | <222> (2502) . . . (2669) |
| | <223> ITR |
| 11 | <213> Artificial Sequence |
| | <223> constructed sequence |
| | <221> misc_feature |
| | <222> (1) . . . (168) |
| | <223> ITR |
| | <221> misc_feature |
| | <222> (206) . . . (1004) |
| | <223> CYP26A1 - 1K promoter |
| | <221> misc_feature |
| | <222> (1013) . . . (2107) |
| | <223> PCS7-8L CDS |
| | <221> misc_feature |
| | <222> (2118) . . . (2332) |
| | <223> bGH Poly A |
| | <221> misc_feature |
| | <222> (2382) . . . (2549) |
| | <223> ITR |
| 12 | <213> Artificial Sequence |
| | <223> constructed sequence |
| | <221> misc_feature |
| | <222> (1) . . . (168) |
| | <223> ITR |
| | <221> misc_feature |
| | <222> (206) . . . (269) |
| | <223> TBG-S1-F64 promoter |
| | <221> misc_feature |
| | <222> (281) . . . (1375) |
| | <223> PCS7-8 CDS |
| | <221> misc_feature |
| | <222> (1386) . . . (1600) |
| | <223> bGH Poly A |
| | <221> misc_feature |
| | <222> (1650) . . . (1817) |
| | <223> ITR |
| 13 | <213> Artificial Sequence |
| | <223> constructed sequence |
| | <221> misc_feature |
| | <222> (1) . . . (168) |
| | <223> ITR |
| | <221> misc_feature |
| | <222> (206) . . . (318) |
| | <223> TBG-S1-F113 promoter |
| | <221> misc_feature |
| | <222> (330) . . . (1424) |
| | <223> PCS7-8 CDS |
| | <221> misc_feature |
| | <222> (1435) . . . (1649) |
| | <223> bGH Poly A |
| | <221> misc_feature |
| | <222> (1699) . . . (1866) |
| | <223> ITR |
| 14 | <213> Artificial Sequence |
| | <223> constructed sequence |
| | <221> misc_feature |
| | <222> (1) . . . (168) |
| | <223> ITR |
| | <221> misc_feature |
| | <222> (206) . . . (345) |
| | <223> TBG-S1-F140 |
| | <221> misc_feature |
| | <222> (357) . . . (1451) |
| | <223> PCS7-8 CDS |
| | <221> misc_feature |
| | <222> (1462) . . . (1676) |
| | <223> bGH PolyA |
| | <221> misc_feature |
| | <222> (1726) . . . (1893) |
| | <223> ITR |
| 15 | <213> Artificial sequence |
| | <223> PCSK7-8L.197 |
| | <221> CDS |
| | <222> (1089) . . . (2183) |
| | <223> M2PCSK9 coding sequence |
| 16 | <213> Artificial sequence |
| | <223> Synthetic Construct |
| 17 | <221> CDS |
| | <222> (1) . . . (2214) |
| 19 | <221> CDS |
| | <222> (1) . . . (2214) |
| 21 | <221> CDS |
| | <222> (1) . . . (2214) |
| 23 | <221> CDS |
| | <222> (1) . . . (2217) |
| 25 | <213> Artificial Sequence |
| | <223> constructed sequence |

What is claimed is:

1. A gene targeting nuclease expression cassette comprising a nucleic acid comprising a nuclease coding sequence which is operably linked to regulatory sequences which direct expression of the nuclease following delivery to a host cell having a sequence to which the nuclease is targeted, wherein the regulatory sequences comprise a promoter which has low transcriptional activity.

2. The nuclease expression cassette according to claim 1, wherein the promoter is a liver-specific promoter.

3. The nuclease expression cassette according to claim 1 or claim 2, wherein the promoter is a TBG-S1 promoter variant.

4. The nuclease expression cassette according to claim 3, wherein the TBG-S1 variant is truncated at the 5′ end or the 3′ end, or both.

5. The nuclease expression cassette according to claim 4, wherein the TBG-S1 variant is truncated at the 5′ end.

6. The nuclease expression cassette according to any one of claims 1 to 5, wherein the promoter is TBG-S1-F64 (SEQ ID NO: 6).

7. The nuclease expression cassette according to any one of claims 1 to 5, wherein the promoter is TBG-S1-F113 (SEQ ID NO: 7).

8. The nuclease expression cassette according to any one of claims 1 to 5, wherein the promoter is TBG-S1-F140 (SEQ ID NO: 8).

9. The nuclease expression cassette according to claim 1 or claim 2, wherein the promoter is a CCL16 promoter.

10. The nuclease expression cassette according to claim 1 or claim 2, wherein the promoter is a SCLC22A9 promoter.

11. The nuclease expression cassette according to claim 1 or claim 2, wherein the promoter is a CYP26A1 promoter.

12. The nuclease expression cassette according to any one of claims 1 to 11, wherein the nuclease is a meganuclease, a CRISPR/Cas nuclease, zinc finger nuclease, or TALEN.

13. The nuclease expression cassette according to any one of claims 1 to 11, wherein the nuclease is a meganuclease.

14. A pharmaceutical composition comprising a nuclease expression cassette according to any one of claims 1 to 13 and one or more of a carrier, suspending agent, and/or excipient.

15. The pharmaceutical composition according to claim 14, wherein the expression is in a non-viral delivery system.

16. The pharmaceutical composition according to claim 15, wherein the non-viral delivery system is a lipid nanoparticle.

17. A viral vector comprising nuclease expression cassette according to any one of claims 1 to 13.

18. A recombinant AAV useful for gene editing comprising an AAV capsid and a vector genome packaged in the AAV capsid, said vector genome comprising:
(a) an expression cassette according to any of claims 1 to 13, and
(b) AAV inverted terminal repeat required for packaging the expression cassette into the capsid.

19. A composition comprising a viral vector according to claim 17 or a recombinant AAV according to claim 18 and one or more of a carrier, diluent, and/or excipient.

20. A method for editing a targeted gene, said method comprising delivering a nuclease expression cassette according to any one of claims 1 to 13, a composition according to any of claims 14 to 16, a viral vector according to claim 17, or an rAAV according to claim 18.

21. A method for reducing off-target activity of a gene targeting nuclease, said method comprising delivering a nuclease expression cassette according to any one of claims 1 to 13, a composition according to any of claims 14 to 16, a viral vector according to claim 17, or an rAAV according to claim 18.

22. A promoter comprising the sequence of SEQ ID NO: 6, SEQ ID NO: 7, or SEQ ID NO: 8.

23. An expression cassette comprising the promoter of claim 22.

24. An expression vector comprising the promoter of claim 22.
